(19) United States
(12) Patent Application Publication
(10) Pub. No.: US 2026/0035439 A1
(43) Pub. Date: Feb. 5, 2026

(54) Anti-Sars-COV-2 Antibodies and Uses Thereof

(71) Applicant: AbbVie Inc., North Chicago, IL (US)

(72) Inventors: Danyal Butt, Shrewsbury, MA (US); Ivan R. Correia, Winchester, MA (US)

(73) Assignee: AbbVie Inc., North Chicago, IL (US)

(21) Appl. No.: 19/284,861

(22) Filed: Jul. 30, 2025

Related U.S. Application Data
(60) Provisional application No. 63/677,380, filed on Jul. 30, 2024.

(51) Int. Cl. C07K 16/10; A61P 31/14

(52) U.S. Cl. CPC C07K 16/1003; A61P 31/14; C07K 2317/565; C07K 2317/76; C07K 2317/92

(57) ABSTRACT

Provided herein are monoclonal antibodies that specifically bind to an anti-SARS-CoV-2 spike(S) protein, and methods of using said antibodies.

### RELATED APPLICATION

This application claims the benefit of U.S. Provisional Application No. 63/677,380, filed Jul. 30, 2024, the contents in which are incorporated herein it its entirety.

### FIELD

This disclosure relates to antibodies, and fragments thereof, that specifically bind to the spike(S) protein of a SARS-COV-2 virus and methods related thereto.

### BACKGROUND

Severe acute respiratory syndrome coronavirus 2 (SARS-COV-2; alternatively referred to as “2019 novel coronavirus”, or “2019-nCOV”) is a positive-sense single-stranded ribonucleic (RNA) virus of the Coronaviridae family. SARS-COV-2 infection is highly contagious and causes a broad range of symptoms, known as coronavirus disease 2019 (COVID-19). COVID-19 disease ranges in severity, from mild or no symptoms to serious complications that may be fatal. Adults over 65 years old, individuals who have underlying medical conditions, or individuals who are immunosuppressed often experience more severe or fatal forms of COVID-19.

More than six percent of the adult population in the United States has an immunosuppressive condition. Many immunocompromised patients are not candidates for vaccination or do not elicit an immune response and account for a significant portion of hospitalizations and deaths (Rubin et al NEJM Audio interview; Goldman et al. J Immunother Cancer. 2021 June;9 (6): e002630; Harpaz R, Dahl R M, Dooling K L, 2013. JAMA 2016; 316:2547-8; Singson et. al. MMWR (CDC) Weekly/Jul. 8, 2022/71 (27);878-884). Accordingly, development of pre-exposure prophylaxis (PrEP) therapy against SARS-COV-2 infection in immunocompromised population remains a priority as there are limited agents that are effective for this population due to mutational escape (Patel et. al., (2020). Emerging Infectious Diseases,26 (8), 1720-1730). Therefore, as COVID-19 infections persist and SARS-COV-2 variants evolve, an unmet need remains for prophylactic and therapeutic treatment options in immunocompromised individuals.

### SUMMARY OF THE INVENTION

In one aspect, provided herein is a monoclonal antibody that specifically binds to an anti-SARS-COV-2 spike(S) protein, wherein the antibody comprises: a heavy chain variable region comprising a CDRH1 domain comprising the amino acid sequence of SEQ ID NO: 1, a CDRH2 domain comprising the amino acid sequence of SEQ ID NO: 2, and a CDRH3 domain comprising the amino acid sequence of SEQ ID NO:3; and a light chain variable region comprising a CDRL1 domain comprising the amino acid sequence of SEQ ID NO: 4, a CDRL2 domain comprising the amino acid sequence of SEQ ID NO: 5, and a CDRL3 domain comprising the amino acid sequence of SEQ ID NO: 6. In some embodiments, the heavy chain variable region comprises the amino acid sequence of SEQ ID NO: 7. In some embodiments, the light chain variable region comprises the amino acid sequence of SEQ ID NO: 8. In one embodiment, the heavy chain comprises the amino acid sequence of SEQ ID NO: 9. In one embodiment, the light chain comprises the amino acid sequence of SEQ ID NO: 10.

In another aspect, provided herein is a monoclonal antibody that specifically binds to an anti-SARS-COV-2 spike(S) protein, wherein the antibody comprises: a heavy chain variable region comprising a CDRH1 domain comprising the amino acid sequence of SEQ ID NO: 11, a CDRH2 domain comprising the amino acid sequence of SEQ ID NO: 12, and a CDRH3 domain comprising the amino acid sequence of SEQ ID NO: 13; and a light chain variable region comprising a CDRL1 domain comprising the amino acid sequence of SEQ ID NO: 14, a CDRL2 domain comprising the amino acid sequence of SEQ ID NO: 15, and a CDRL3 domain comprising the amino acid sequence of SEQ ID NO: 16. In some embodiments, the heavy chain variable region comprises the amino acid sequence of SEQ ID NO: 17. In some embodiments, the light chain variable region comprises the amino acid sequence of SEQ ID NO: 18. In one embodiment, the heavy chain comprises the amino acid sequence of SEQ ID NO: 19. In one embodiment, the light chain comprises the amino acid sequence of SEQ ID NO: 20.

In a further aspect, provided herein is a monoclonal antibody that specifically binds to an anti-SARS-COV-2 spike(S) protein, wherein the antibody comprises: a heavy chain variable region comprising a CDRH1 domain comprising the amino acid sequence of SEQ ID NO: 21, a CDRH2 domain comprising the amino acid sequence of SEQ ID NO: 22, and a CDRH3 domain comprising the amino acid sequence of SEQ ID NO: 23; and a light chain variable region comprising a CDRL1 domain comprising the amino acid sequence of SEQ ID NO: 24, a CDRL2 domain comprising the amino acid sequence of SEQ ID NO: 25, and a CDRL3 domain comprising the amino acid sequence of SEQ ID NO: 26. In some embodiments, the heavy chain variable region comprises the amino acid sequence of SEQ ID NO: 27 and a light chain variable region comprises the amino acid sequence of SEQ ID NO: 28. In one embodiment, the monoclonal antibody comprises a heavy chain comprising the amino acid sequence of SEQ ID NO: 29 and a light chain comprising the amino acid sequence of SEQ ID NO: 30.

In some embodiments of any of the aspects provided herein, the monoclonal antibody is an IgG antibody. In some embodiments, the IgG antibody is an IgG1 or IgG4.

In a further aspect, provided herein is a pharmaceutical composition comprising (i) a monoclonal antibody comprising a heavy chain variable region comprising a CDRH1 domain comprising the amino acid sequence of SEQ ID NO: 1, a CDRH2 domain comprising the amino acid sequence of SEQ ID NO: 2, and a CDRH3 domain comprising the amino acid sequence of SEQ ID NO:3; and a light chain variable region comprising a CDRL1 domain comprising the amino acid sequence of SEQ ID NO: 4, a CDRL2 domain comprising the amino acid sequence of SEQ ID NO: 5, and a CDRL3 domain comprising the amino acid sequence of SEQ ID NO: 6; and (ii) a monoclonal antibody comprising a heavy chain variable region comprising a CDRH1 domain comprising the amino acid sequence of SEQ ID NO: 11, a CDRH2 domain comprising the amino acid sequence of SEQ ID NO: 12, and a CDRH3 domain comprising the amino acid sequence of SEQ ID NO: 13; and a light chain variable region comprising a CDRL1 domain comprising the amino acid sequence of SEQ ID NO: 14, a CDRL2 domain comprising the amino acid sequence of SEQ ID NO: 15, and a CDRL3 domain comprising the amino acid sequence of SEQ ID NO: 16.

In another aspect, provided herein is a pharmaceutical composition comprising (i) a monoclonal antibody comprising a heavy chain variable region comprising a CDRH1 domain comprising the amino acid sequence of SEQ ID NO: 1, a CDRH2 domain comprising the amino acid sequence of SEQ ID NO: 2, and a CDRH3 domain comprising the amino acid sequence of SEQ ID NO:3; and a light chain variable region comprising a CDRL1 domain comprising the amino acid sequence of SEQ ID NO: 4, a CDRL2 domain comprising the amino acid sequence of SEQ ID NO: 5, and a CDRL3 domain comprising the amino acid sequence of SEQ ID NO: 6; and; and (ii) a monoclonal antibody comprising a heavy chain variable region comprising a CDRH1 domain comprising the amino acid sequence of SEQ ID NO: 21, a CDRH2 domain comprising the amino acid sequence of SEQ ID NO: 22, and a CDRH3 domain comprising the amino acid sequence of SEQ ID NO: 23; and a light chain variable region comprising a CDRL1 domain comprising the amino acid sequence of SEQ ID NO: 24, a CDRL2 domain comprising the amino acid sequence of SEQ ID NO: 25, and a CDRL3 domain comprising the amino acid sequence of SEQ ID NO: 26.

In another aspect, provided herein is a pharmaceutical composition comprising (i) a monoclonal antibody comprising a heavy chain variable region comprising a CDRH1 domain comprising the amino acid sequence of SEQ ID NO: 11, a CDRH2 domain comprising the amino acid sequence of SEQ ID NO: 12, and a CDRH3 domain comprising the amino acid sequence of SEQ ID NO: 13; and a light chain variable region comprising a CDRL1 domain comprising the amino acid sequence of SEQ ID NO: 14, a CDRL2 domain comprising the amino acid sequence of SEQ ID NO: 15, and a CDRL3 domain comprising the amino acid sequence of SEQ ID NO: 16; and (ii) a monoclonal antibody comprising a heavy chain variable region comprising a CDRH1 domain comprising the amino acid sequence of SEQ ID NO: 21, a CDRH2 domain comprising the amino acid sequence of SEQ ID NO: 22, and a CDRH3 domain comprising the amino acid sequence of SEQ ID NO: 23; and a light chain variable region comprising a CDRL1 domain comprising the amino acid sequence of SEQ ID NO: 24, a CDRL2 domain comprising the amino acid sequence of SEQ ID NO: 25, and a CDRL3 domain comprising the amino acid sequence of SEQ ID NO: 26.

In some embodiments, the pharmaceutical composition provided herein is for use in treating a SARS-COV-2 infection.

In another aspect, provided is a method of controlling levels of a SARS-COV-2 virus in a subject comprising administering an effective amount of at least one monoclonal antibody provided herein to a human subject in need thereof.

In another aspect, provided is a method of controlling levels of a SARS-COV-2 virus in a human subject comprising administering an effective amount of a pharmaceutical composition provided herein to a human subject in need thereof.

In another aspect, provided is a method of reducing and maintaining low levels of a SARS-COV-2 virus in a human subject comprising administering an effective amount of at least one monoclonal antibody provided herein to a human subject in need thereof.

In another aspect, provided is a method of reducing and maintaining low levels of a SARS-COV-2 virus in a human subject comprising administering an effective amount of a pharmaceutical composition provided herein to a human subject in need thereof.

In another, provided is a method of controlling levels of a SARS-COV-2 virus in a human subject comprising administering an effective amount of a monoclonal antibody to a human subject in need thereof, wherein the antibody specifically binds to an anti-SARS-COV-2 spike(S) protein and comprises a heavy chain variable region comprising a CDRH1 domain comprising the amino acid sequence of SEQ ID NO: 1, a CDRH2 domain comprising the amino acid sequence of SEQ ID NO: 2, and a CDRH3 domain comprising the amino acid sequence of SEQ ID NO:3; and a light chain variable region comprising a CDRL1 domain comprising the amino acid sequence of SEQ ID NO: 4, a CDRL2 domain comprising the amino acid sequence of SEQ ID NO: 5, and a CDRL3 domain comprising the amino acid sequence of SEQ ID NO: 6. In some embodiments, the heavy chain variable region comprises the amino acid sequence of SEQ ID NO: 7. In some embodiments, the light chain variable region comprises the amino acid sequence of SEQ ID NO: 8. In some embodiments, the heavy chain comprises the amino acid sequence of SEQ ID NO: 9. In some embodiments, the light chain comprises the amino acid sequence of SEQ ID NO: 10.

In another aspect, a method of controlling levels of a SARS-COV-2 virus in a human subject comprising administering an effective amount of a monoclonal antibody to a human subject in need thereof, wherein the antibody specifically binds to an anti-SARS-COV-2 spike(S) protein and comprises a heavy chain variable region comprising a CDRH1 domain comprising the amino acid sequence of SEQ ID NO: 11, a CDRH2 domain comprising the amino acid sequence of SEQ ID NO: 12, and a CDRH3 domain comprising the amino acid sequence of SEQ ID NO: 13; and a light chain variable region comprising a CDRL1 domain comprising the amino acid sequence of SEQ ID NO: 14, a CDRL2 domain comprising the amino acid sequence of SEQ ID NO: 15, and a CDRL3 domain comprising the amino acid sequence of SEQ ID NO: 16. In some embodiments, the heavy chain variable region comprises the amino acid sequence of SEQ ID NO: 17. In some embodiments, the light chain variable region comprises the amino acid sequence of SEQ ID NO: 18. In one embodiment, the heavy chain comprises the amino acid sequence of SEQ ID NO: 19. In one embodiment, the light chain comprises the amino acid sequence of SEQ ID NO: 20.

In another aspect, provided herein is a method of controlling levels of a SARS-CoV-2 virus in a human subject comprising administering an effective amount of a monoclonal antibody to a human subject in need thereof, wherein the antibody specifically binds to an anti-SARS-COV-2 spike(S) protein and comprises a heavy chain variable region comprising a CDRH1 domain comprising the amino acid sequence of SEQ ID NO: 21, a CDRH2 domain comprising the amino acid sequence of SEQ ID NO: 22, and a CDRH3 domain comprising the amino acid sequence of SEQ ID NO: 23; and a light chain variable region comprising a CDRL1 domain comprising the amino acid sequence of SEQ ID NO: 24, a CDRL2 domain comprising the amino acid sequence of SEQ ID NO: 25, and a CDRL3 domain comprising the amino acid sequence of SEQ ID NO: 26. In some embodiments, the heavy chain variable region comprises the amino acid sequence of SEQ ID NO: 27. In some embodiments, the light chain variable region comprises the amino acid sequence of SEQ ID NO: 28. In one embodiment, the heavy chain comprises the amino acid sequence of SEQ ID NO: 29. In one embodiment, the light chain comprises the amino acid sequence of SEQ ID NO: 30.

In some embodiments, the human subject is immunocompromised.

### BRIEF DESCRIPTION OF THE DRAWINGS

FIG. 1 depicts a schematic of an epitope mapping procedure by hydrogen/deuterium exchange mass spectrometry (HDX-MS).

### DETAILED DESCRIPTION

### Definitions

Unless otherwise defined herein, scientific and technical terms used in connection with the present disclosure shall have meanings that are commonly understood by those of ordinary skill in the art.

The term “antibody”, as used herein, is intended to refer to immunoglobulin molecules comprised of four polypeptide chains, two heavy (H) chains and two light (L) chains inter-connected by disulfide bonds. Each heavy chain is comprised of a heavy chain variable region (abbreviated herein as HCVR or VH) and a heavy chain constant region. The heavy chain constant region is comprised of three domains, CH1, CH2 and CH3. Each light chain is comprised of a light chain variable region (abbreviated herein as LCVR or VL) and a light chain constant region. The light chain constant region is comprised of one domain, CL. The VH and VL regions can be further subdivided into regions of hypervariability, termed complementarity determining regions (CDR), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, FR4.

Also contemplated are antigen-binding fragments. The binding fragments of the disclosure include those that specifically bind to a spike protein of an anti-SARS-COV-2 virus, particularly the receptor binding domain (RBD) of the spike(S) protein. Examples of antigen-binding fragments include by way of example and not limitation, Fab, Fab′, F(ab′)₂, Fv fragments, and single domain fragments.

A Fab fragment contains the constant domain of the light chain and the first constant domain (CH1) of the heavy chain. Fab′ fragments differ from Fab fragments by the addition of a few residues at the carboxyl terminus of the heavy chain CH1 domain including one or more cysteines from the antibody hinge region. Fab′ fragments are produced by cleavage of the disulfide bond at the hinge cysteines of the F(ab′) 2 pepsin digestion product. Additional chemical couplings of antibody fragments are known to those of ordinary skill in the art. Fab and F(ab′) 2 fragments lack the Fragment crystallizable (Fc) region of an intact antibody, clear more rapidly from the circulation of animals, and may have less non-specific tissue binding than an intact antibody (see, e.g., Wahl et al., 1983, J. Nucl. Med. 24:316).

As is commonly understood in the art, an “Fc” region is the Fragment crystallizable constant region of an antibody not comprising an antigen-specific binding region. In IgG, IgA and IgD antibody isotypes, the Fc region is composed of two identical protein fragments, derived from the second and third constant domains (CH2 and CH3 domains, respectively) of the two heavy chains of an antibody. IgM and IgE Fc regions contain three heavy chain constant domains (CH2, CH3, and CH4 domains) in each polypeptide chain.

An “Fv” fragment is the minimum fragment of an antibody that contains a complete target recognition and binding site. This region consists of a dimer of one heavy and one light chain variable domain in a tight, non-covalent association (VH-VL dimer). It is in this configuration that the three CDRs of each variable domain interact to define a target binding site on the surface of the VH-VL dimer. Often, the six CDRs confer target binding specificity to the antibody. However, in some instances even a single variable domain (or half of an Fv comprising only three CDRs specific for a target) can have the ability to recognize and bind target, although at a lower affinity than the entire binding site.

“Single domain fragments” are composed of a single VH or VL domains which exhibit sufficient affinity to an anti-SARS-COV-2 virus S protein. In a specific embodiment, the single domain fragment is camelized (See, e.g., Riechmann, 1999, Journal of Immunological Methods 231:25-38).

A “single-chain Fv” or “scFv” comprises the VH and VL domains of an antibody, where these domains are present in a single polypeptide chain. Generally, the scFv polypeptide further comprises a polypeptide linker between the VH and VL domains which enables the scFv to form a structure favorable for target binding.

As used herein, the term “diabody” refers to a dimer comprising two scFv molecules that are covalently or noncovalently attached. The scFv molecule may further be conjugated to other protein(s) or an active fragment thereof. In such an instance, a diabody would contain a dimer of a fusion protein, each containing an scFv.

As used herein, the term “subject” refers to a human which is to be the recipient of a particular treatment. The terms “subject” and “patient” are used interchangeably herein in reference to a human subject.

As used herein the term, the term “immunocompromised” refers to a subject that has an impaired immune system. The immunocompromised condition of a subject may be due to one or more defects or dysfunctions of the immune system and/or to other factors (e.g., treatment with an immunosuppressive agent).

As used herein, “an effective amount” refers to the amount of an active agent (e.g., anti-SARS-COV-2 spike protein antibody) that confers a therapeutic effect on the subject, either alone or in combination with one or more other active agents. In some embodiments, an effective amount of an anti-SARS-COV-2 spike protein antibody treats or prevents a SARS-COV-2 infection in a human subject.

As used herein, the terms “treating” or “treatment” each refers to controlling infectious viral levels, reducing infectious viral levels, or maintaining low infectious viral levels in a subject. The treatment may be effective to ameliorate or alleviate a viral infection or one or more symptoms associated with the infection, such as a SARS-COV-2 infection.

The term “prophylactic” or “prophylactic treatment” as used herein, refers to administering an anti-SARS-COV-2 spike protein antibody to a subject who is at risk of a SARS-COV-2 viral infection.

The term “subject in need thereof” in the context of prophylactic treatment refers to a subject vulnerable to or at risk of a SARS-COV-2 infection, including subjects at risk of developing symptoms associated with a SARS-COV-2 infection. Such subjects include but are not limited to those prone to symptoms associated with a SARS-COV-2 viral infection (e.g., individuals who may have mild to moderate symptoms or moderate to severe symptoms that may progress to hospitalization or death); and/or those who have been exposed to or are at high risk of exposure to other individuals infected with a SARS-COV-2 virus. Individuals at risk of SARS-COV-2 infection further include subjects who are ineligible for SARS-COV-2 vaccination, who are unvaccinated against SARS-CoV-2, who have not received all available booster doses of a SARS-COV-2 vaccine, or who have ineffective immune responses to a SARS-COV-2 vaccine. The subject may be either immunocompetent or immunocompromised. In certain embodiments, the subject in need of preventative or prophylactic treatment is immunocompromised.

As used herein, the term “Severe Acute Respiratory Syndrome Coronavirus 2 (SARS-COV-2)” refers to all strain variants of SARS-COV-2 that are capable of infecting humans, including those that cause symptoms or disease (e.g., COVID-19) in humans.

As used herein, the term “specifically binds” refers to the ability of an antibody to bind to an antigen with a KD of at least about 1×10⁻⁶ M, 1×10⁻⁷ M, 1×10⁻⁸ M, 1×10⁻⁹ M, 1×10⁻¹⁰ M, 1×10⁻¹¹ M, 1×10⁻¹² M, 1×10⁻¹³ M, 1×10⁻¹⁴ M or more, and/or bind to an antigen with an affinity that is at least two-fold greater than its affinity for a nonspecific antigen.

The term “vector”, as used herein, refers to a nucleic acid molecule capable of transporting another nucleic acid to which it has been linked. One type of vector is a “plasmid”, which refers to a circular double stranded DNA loop into which additional DNA segments may be ligated. Another type of vector is a viral vector, wherein additional DNA segments may be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) can be integrated into the genome of a host cell upon introduction into the host cell, and thereby are replicated along with the host genome. Moreover, certain vectors are capable of directing the expression of genes to which they are operatively linked. Such vectors are referred to herein as “recombinant expression vectors” (or simply, “expression vectors”). In general, expression vectors of utility in recombinant DNA techniques are often in the form of plasmids. In the present specification, “plasmid” and “vector” may be used interchangeably as the plasmid is the most commonly used form of vector. However, the invention is intended to include such other forms of expression vectors, such as viral vectors (e.g., replication defective retroviruses, adenoviruses, and adeno-associated viruses), which serve equivalent functions.

Various aspects of the invention are described in further detail in the following subsections.

### Anti-SARS-COV-2 Spike Antibodies

Disclosed herein are antibodies, and fragments thereof, that specifically bind to the spike(S) protein of a SARS-COV-2 virus. The disclosed antibodies bind conserved regions of the SARS-COV-2 spike protein, retain potent neutralization against a wide-range of known SARS-COV-2 variants, and have an enhanced half-life, making them amenable for use in treating or preventing infections by a SARS-COV-2 virus. Further, by generating a library of antibodies that target different epitopes on the spike protein, antibodies having distinct mechanisms of action were identified herein. The identified antibodies include both ACE-2 competitive and non-ACE-2 competitive antibodies.

To investigate whether antibody bound Anti SARS-COV-2 RBD (receptor binding domain) complexes would be blocked from binding to its receptor ACE-2, binding studies were conducted using recombinant SARS-COV-2 RBD WT and ACE-2 receptor proteins. The ability of captured Anti SARS-COV-2 antibodies bound to SARS-COV-2 RBD WT complexes to bind to human recombinant ACE-2 receptor was assessed by surface plasmon resonance (SPR).

Anti SARS-COV-2 antibodies were captured via Goat Anti Human Fc as ligands, on a Biacore chip (Cytiva) and recombinant SARS-COV-2 RBD WT was injected as analyte, in solution, and a positive binding event was monitored during the association phase. Sequentially, an excess of human recombinant ACE-2 receptor was injected over the bound Anti SARS-COV-2 Mab/SARS-COV-2 RBD WT complex and the ability of ACE-2 binding was monitored in time.

Anti SARS-COV-2 antibodies that shared overlapping epitopes with ACE-2 in binding to SARS-COV-2 RBD WT displayed no binding events in the presence of human recombinant ACE-2 receptor, thereby completely blocking (competing) with ACE-2 active binding sites (in the case of ABBV-1403 (TPP-16613), PR-2246604-AZD-3152, PR-2134258-Bebtelovimab).

Anti SARS-COV-2 antibodies that have non-overlapping epitopes with ACE-2 in binding to SARS-COV-2 RBD WT displayed a positive binding event upon injection of the human recombinant ACE-2 receptor, not blocking or non-competing with ACE-2 binding sites (in the case of ABBV-2412 (TPP-14584), PR-2208877 (TPP-16124), PR-1970409-S309).

Accordingly, additionally provided are compositions that include combinations of two or more antibodies with non-overlapping epitopes. Treatment with combinations of the disclosed antibodies maximizes the footprint of the antibodies on the receptor binding domain of the spike protein, thereby enhancing the potential for clinical benefit in subjects, such as immunocompromised subjects, infected with or at risk of infection with a SARS-COV-2 virus.

Severe Acute Respiratory Syndrome Coronavirus 2 (SARS-COV-2; alternatively referred to as 2019 novel coronavirus (2019-nCOV) or human coronavirus 2019 (HCoV-19 or hCoV-19) is a Baltimore class IV positive-sense single-stranded RNA virus that is contagious in humans and other mammals. SARS-COV-2 was initially identified from the Chinese city of Wuhan in December 2019. The pandemic disease caused by SARS-CoV-2 was named by the World Health Organization (WHO) as COVID-19 (Coronavirus Disease 2019). The first genome sequence of a SARS-COV-2 isolate (also referred to as 2019 nCOV or Wuhan-Hu-1) was deposited in GenBank on Jan. 12, 2020 by investigators from the Chinese CDC in Beijing.

Each SARS-COV-2 virion is 50-200 nm in diameter. Like other coronaviruses, SARS-COV-2 has four structural proteins, known as the S (spike), E (envelope), M (membrane), and N (nucleocapsid) proteins; the N protein holds the RNA genome, and the S, E, and M proteins together create the viral envelope.

The spike protein is responsible for allowing the virus to attach to and fuse with the membrane of a host cell; specifically, its S1 subunit contains a receptor binding domain that is responsible for attachment to a host cell and the S2 subunit is responsible for membrane fusion with the host cell. The receptor binding domain (RBD) of the S1 subunit of the spike protein binds to receptor angiotensin converting enzyme 2 (ACE2) on human cells with sufficient affinity to use the ACE-2 receptor as a mechanism of cell entry (Tai, W., et al. (2020). Cellular & molecular immunology, 17 (6), 613-620). The amino acid sequence of the SARS-COV-2 spike protein and variants thereof are further described at, for example, Uniprot Accession No. PODTC2. The amino acid sequence of the SARS-COV-2 spike protein (SEQ ID NO: 31), is additionally provided below, with residues corresponding to the spike protein RBD (residues 319-541 of SEQ ID NO: 31) underlined and bolded (See also Yan, R., et al. (2020). Science, 367 (6485), 1444-1448):

SARS-COV-2 Spike Protein (Uniprot Checksum: B17BE6D9F1C4EA34); RBD domain, corresponding to residues 319-541, is underlined and bolded:

(SEQ ID NO: 31)
MFVFLVLLPLVSSQCVNLTTRTQLPPAYTNSFTRGVYYPDKVFRSSVLHS
TQDLFLPFFSNVTWFHAIHVSGTNGTKRFDNPVLPFNDGVYFASTEKSNI
IRGWIFGTTLDSKTQSLLIVNNATNVVIKVCEFQFCNDPFLGVYYHKNNK
SWMESEFRVYSSANNCTFEYVSQPFLMDLEGKQGNFKNLREFVFKNIDGY
FKIYSKHTPINLVRDLPQGFSALEPLVDLPIGINITRFQTLLALHRSYLT
PGDSSSGWTAGAAAYYVGYLQPRTFLLKYNENGTITDAVDCALDPLSETK
CTLKSFTVEKGIYQTSNFRVQPTESIVRFPNITNLCPFGEVFNATRFASV
YAWNRKRISNCVADYSVLYNSASFSTFKCYGVSPTKLNDLCFTNVYADSF
VIRGDEVRQIAPGQTGKIADYNYKLPDDFTGCVIAWNSNNLDSKVGGNYN
YLYRLFRKSNLKPFERDISTEIYQAGSTPCNGVEGFNCYFPLQSYGFQPT
NGVGYQPYRVVVLSFELLHAPATVCGPKKSTNLVKNKCVNFNFNGLTGTG
VLTESNKKFLPFQQFGRDIADTTDAVRDPQTLEILDITPCSFGGVSVITP
GTNTSNQVAVLYQDVNCTEVPVAIHADQLTPTWRVYSTGSNVFQTRAGCL
IGAEHVNNSYECDIPIGAGICASYQTQTNSPRRARSVASQSIIAYTMSLG
AENSVAYSNNSIAIPTNFTISVTTEILPVSMTKTSVDCTMYICGDSTECS
NLLLQYGSFCTQLNRALTGIAVEQDKNTQEVFAQVKQIYKTPPIKDFGGF
NFSQILPDPSKPSKRSFIEDLLFNKVTLADAGFIKQYGDCLGDIAARDLI
CAQKFNGLTVLPPLLTDEMIAQYTSALLAGTITSGWTFGAGAALQIPFAM
QMAYRFNGIGVTQNVLYENQKLIANQFNSAIGKIQDSLSSTASALGKLQD
VVNQNAQALNTLVKQLSSNFGAISSVLNDILSRLDKVEAEVQIDRLITGR
LQSLQTYVTQQLIRAAEIRASANLAATKMSECVLGQSKRVDFCGKGYHLM
SFPQSAPHGVVFLHVTYVPAQEKNFTTAPAICHDGKAHFPREGVFVSNGT
HWFVTQRNFYEPQIITTDNTFVSGNCDVVIGIVNNTVYDPLQPELDSFKE
ELDKYFKNHTSPDVDLGDISGINASVVNIQKEIDRLNEVAKNLNESLIDL
QELGKYEQYIKWPWYIWLGFIAGLIAIVMVTIMLCCMTSCCSCLKGCCSC
GSCCKFDEDDSEPVLKGVKLHYT

Provided herein are antibodies, or antigen-binding fragments thereof, that specifically bind to an anti-SARS-COV-2 spike protein.

The anti-SARS-COV-2 spike antibody can be an ACE-2 competitive or non-ACE-2 competitive antibody. These represent distinct mechanisms of action arising from different epitopes on the spike protein. In some embodiments, the antibody is an ACE-2 competitive antibody (i.e., an antibody that targets the spike receptor-binding domain in a manner that competes for binding to ACE-2). In alternative embodiments, the antibody is a non-ACE-2 competitive antibody (i.e., an antibody that targets the spike receptor-binding domain in a manner that does not compete for binding to ACE-2).

In some embodiments, the antibody is a non-ACE-2 competitive antibody. In one embodiment, the non-ACE-2 competitive antibody binds to an epitope comprising three peptide regions of the spike protein: ¹²⁴DSKVGGNYNYL¹³⁴ (SEQ ID NO: 32), ¹⁵⁰ISTE¹⁵³ (SEQ ID NO: 33), and ¹⁷⁷YGF¹⁷⁹ (SEQ ID NO: 34). In some embodiments, non-ACE-2 competitive antibody further binds to an allosteric epitope comprising the following peptide region of the spike protein: ⁹¹QIAPGQTGNIADYN¹⁰⁴ (SEQ ID NO: 35). See, for example, the epitope of antibody TPP-14584 described in Example 3.

In some embodiments, the antibody is an ACE-2 competitive antibody. In one embodiment, the ACE-2 competitive antibody binds to an epitope comprising three peptide regions of the spike protein: ⁹¹QIAPGQTGNIADYN¹⁰⁴ (SEQ ID NO: 40), ¹³⁶RLF¹³⁸ (SEQ ID NO: 41), and ¹⁵⁴IYQAGNKPCNGVAGFNCYFP¹⁷³ (SEQ ID NO: 42).

In another embodiment, the ACE-2 competitive antibody binds to an epitope comprising three peptide regions of the spike protein: ³¹SVYAWNRKRISNCVAD⁴⁶ (SEQ ID NO: 36), ⁷⁵TNVYADSF⁸² (SEQ ID NO: 37), ¹³²NYL¹³⁴ (SEQ ID NO: 38), and ¹⁵⁰ISTE¹⁵³ (SEQ ID NO: 39).

In some embodiments, the anti-SARS-COV-2 spike antibody includes a heavy chain variable region comprising a CDR1 domain comprising the amino acid sequence of SEQ ID NO: 1, a CDR2 domain comprising the amino acid sequence of SEQ ID NO: 2, and a CDR3 domain comprising the amino acid sequence of SEQ ID NO:3; and a light chain variable region comprising a CDR1 domain comprising the amino acid sequence of SEQ ID NO: 4, a CDR2 domain comprising the amino acid sequence of SEQ ID NO: 5, and a CDR3 domain comprising the amino acid sequence of SEQ ID NO: 6.

In some embodiments, the anti-SARS-COV-2 spike antibody includes a heavy chain variable region comprising the amino acid sequence of SEQ ID NO: 7 and a light chain variable region comprising the amino acid sequence of SEQ ID NO: 8.

In some embodiments, the anti-SARS-COV-2 spike antibody comprises a heavy chain comprising the amino acid sequence of SEQ ID NO: 9 and a light chain comprising the amino acid sequence of SEQ ID NO: 10.

In some embodiments, the anti-SARS-COV-2 spike antibody includes a heavy chain variable region comprising a CDR1 domain comprising the amino acid sequence of SEQ ID NO: 11, a CDR2 domain comprising the amino acid sequence of SEQ ID NO: 12, and a CDR3 domain comprising the amino acid sequence of SEQ ID NO: 13; and a light chain variable region comprising a CDR1 domain comprising the amino acid sequence of SEQ ID NO: 14, a CDR2 domain comprising the amino acid sequence of SEQ ID NO: 15, and a CDR3 domain comprising the amino acid sequence of SEQ ID NO: 16.

In some embodiments, the anti-SARS-COV-2 spike antibody includes a heavy chain variable region comprising the amino acid sequence of SEQ ID NO: 17 and a light chain variable region comprising the amino acid sequence of SEQ ID NO: 18.

In some embodiments, the anti-SARS-COV-2 spike antibody comprises a heavy chain comprising the amino acid sequence of SEQ ID NO: 19 and a light chain comprising the amino acid sequence of SEQ ID NO: 20.

In some embodiments, the anti-SARS-COV-2 spike antibody includes a heavy chain variable region comprising a CDR1 domain comprising the amino acid sequence of SEQ ID NO: 21, a CDR2 domain comprising the amino acid sequence of SEQ ID NO: 22, and a CDR3 domain comprising the amino acid sequence of SEQ ID NO:23; and a light chain variable region comprising a CDR1 domain comprising the amino acid sequence of SEQ ID NO: 24, a CDR2 domain comprising the amino acid sequence of SEQ ID NO: 25, and a CDR3 domain comprising the amino acid sequence of SEQ ID NO: 26.

In some embodiments, the anti-SARS-COV-2 spike antibody includes a heavy chain variable region comprising the amino acid sequence of SEQ ID NO: 27 and a light chain variable region comprising the amino acid sequence of SEQ ID NO: 28.

In some embodiments, the anti-SARS-COV-2 spike antibody comprises a heavy chain comprising the amino acid sequence of SEQ ID NO: 29 and a light chain comprising the amino acid sequence of SEQ ID NO: 30.

In another embodiment, the antibody comprises a heavy chain variable region that comprises an amino acid sequence having at least 95% identity to an anti-SARS-CoV-2 spike antibody herein, e.g., at least 95%, 96%, 97%, 98%, 99%, or 100% identity to an anti-SARS-COV-2 spike antibody herein. In certain embodiments, an antibody comprises a modified heavy chain (HC) variable region comprising an HC variable domain of an anti-SARS-COV-2 spike antibody herein, or a variant thereof, which variant (i) differs from the a antibody in 1, 2, 3, 4 or 5 amino acids substitutions, additions or deletions; (ii) differs from the anti-SARS-COV-2 spike antibody in at most 5, 4, 3, 2, or 1 amino acids substitutions, additions or deletions; (iii) differs from the anti-SARS-COV-2 spike antibody in 1-5, 1-3, 1-2, 2-5 or 3-5 amino acids substitutions, additions or deletions and/or (iv) comprises an amino acid sequence that is at least about 75%, 80%, 85%, 90%, 95%, 96%, 97%, 98% or 99% identical to the anti-SARS-COV-2 spike antibody, wherein in any of (i)-(iv), an amino acid substitution may be a conservative amino acid substitution or a non-conservative amino acid substitution; and wherein the modified heavy chain variable region can have an enhanced biological activity relative to the heavy chain variable region of the anti-SARS-COV-2 spike antibody, while retaining the SARS-COV-2 spike binding specificity of the antibody.

The antibodies of the disclosure are monoclonal, recombinant (i.e., genetically engineered) antibodies. In various embodiments, the antibodies comprise all or a portion of a constant region of an antibody. In some embodiments, the constant region is an isotype selected from: IgA (e.g., IgA1 or IgA2), IgD, IgE, IgG (e.g., IgG1, IgG2, IgG3 or IgG4), and IgM. In specific embodiments, the anti-SARS-COV-2 spike antibodies comprise an IgG constant region. In certain embodiments, the anti-SARS-CoV-2 spike antibodies comprise an IgG1 constant region. As used herein, the “constant region” of an antibody includes the natural constant region, allotypes or variants.

The light constant region of an anti-SARS-COV-2 spike antibody disclosed herein may be a kappa (κ) light region or a lambda (λ) region. A λ light region can be any one of the known subtypes, e.g., λ1, λ2, λ3, or λ4. In some embodiments, the anti-SARS-COV-2 spike antibody comprises a kappa (κ) light region.

In some embodiments, the anti-SARS-COV-2 spike antibody is a monoclonal antibody. A monoclonal antibody is derived from a single clone, including any eukaryotic, prokaryotic, or phage clone, by any means available or known in the art. Monoclonal antibodies useful with the present disclosure can be prepared using a wide variety of techniques known in the art including the use of hybridoma, recombinant, and phage display technologies, or a combination thereof. The term monoclonal antibody, as used herein, is not limited to antibodies produced through hybridoma technology.

In some embodiments, the anti-SARS-COV-2 spike antibody is a human antibody. “Human antibodies” include antibodies having the amino acid sequence of a human immunoglobulin and include antibodies isolated from human immunoglobulin libraries or from animals transgenic for one or more human immunoglobulins and that do not express endogenous functional immunoglobulins. Human antibodies can be made by a variety of methods known in the art including phage display methods using antibody libraries derived from human immunoglobulin sequences.

Anti-SARS-COV-2 spike antibodies of the disclosure include full-length (intact) antibody molecules.

The anti-SARS-COV-2 spike antibodies may be antibodies whose sequences have been modified to alter at least one constant region-mediated biological effector function. For example, the anti-SARS-COV-2 spike antibodies described herein include antibodies that have been modified to acquire or improve at least one constant region-mediated biological effector function relative to an unmodified antibody, e.g., to enhance FcγR interactions (See, e.g., US Patent Appl. No. 2006/0134709) or to enhance the antibody's ability to mediate ADCC. For example, an anti-SARS-COV-2 spike antibody of the disclosure can have a constant region that binds FcγRI, FcγRIIA, FcγRIIB, FcγRIIIA and/or FcγRIIIB with greater affinity than the corresponding unmodified constant region. An anti-SARS-COV-2 spike antibody of the disclosure can be one that has a modified Fc region and mediates an enhanced ADCC response, wherein the ADCC response is enhanced with respect to an antibody having the same variable regions (i.e., VH and VL) and a wild type IgG1 Fc region (i.e., wild type CL, CH1 CH2, and CH3). Fc modifications able to enhance ADCC, such as amino acid sequence mutations, are known in the art, and can include the following sets of mutations: S239D/1332E; F243L/R292P/Y300L/V3051/P396L; S239D/1332E/λ330L; and S298A/E333A/K334A.

In some embodiments, the anti-SARS-COV-2 spike antibodies may be antibodies whose sequences have been modified to alter the half-life of the antibody. For example, the anti-SARS-COV-2 spike antibodies described herein include antibodies that have been modified to extend the half-life of the antibody relative to an unmodified antibody. An anti-SARS-COV-2 spike antibody of the disclosure can be one that has a modified Fc region having an extended half-life with respect to an antibody having the same variable regions (i.e., VH and VL) and a wild type IgG1 Fc region (i.e., wild type CL, CH1 CH2, and CH3). Fc modifications that can extend the half-life of antibodies, such as amino acid sequence mutations, are known in the art. In some such embodiments, the anti-SARS-COV-2 antibody comprises a M428L and a N434S mutation (i.e., an LS mutation).

Anti-SARS-COV-2 antibodies with high affinity for SARS-COV-2 may be desirable for therapeutic and diagnostic uses. Accordingly, the present disclosure contemplates antibodies having a high binding affinity to a SARS-COV-2 spike protein. In specific embodiments, the anti-SARS-COV-2 spike antibodies binds to an SARS-COV-2 spike protein with an affinity of at least about 100 nM, but may exhibit higher affinity, for example, at least about 90 nM, 80 nM, 70 nM, 60 nM, 50 nM, 40 nM, 30 nM, 25 nM, 20 nM, 15 nM, 10 nM, 7 nM, 6 nM, 5 nM, 4 nM, 3 nM, 2 nM, 1 nM, 0.1 nM, 0.01 nM, 0.001 nM or higher. In some embodiments, the antibodies bind SARS-COV-2 spike protein with an affinity in the range of about 1 pM to about 10 nM, of about 100 pM to about 10 nM, about 100 pM to about 1 nM, or an affinity ranging between any of the foregoing values.

In some embodiments, the antibody is a recombinant, IgG monoclonal antibody that binds to a SARS-COV-2 spike protein.

Further provided herein are polynucleotide molecules encoding anti-SARS-CoV-2 spike antibodies or antigen-binding fragments thereof, vectors comprising such polynucleotides, and host cells capable of producing the anti-SARS-COV-2 spike antibodies of the disclosure.

In one aspect, provided herein is a polynucleotide comprising a nucleotide sequence encoding a heavy chain variable region comprising a CDRH1 domain comprising the amino acid sequence of SEQ ID NO: 1, a CDRH2 domain comprising the amino acid sequence of SEQ ID NO: 2, and a CDRH3 domain comprising the amino acid sequence of SEQ ID NO:3; and a light chain variable region comprising a CDRL1 domain comprising the amino acid sequence of SEQ ID NO: 4, a CDRL2 domain comprising the amino acid sequence of SEQ ID NO: 5, and a CDRL3 domain comprising the amino acid sequence of SEQ ID NO: 6. In some embodiments, the heavy chain variable region comprises the amino acid sequence of SEQ ID NO: 7. In some embodiments, the light chain variable region comprises the amino acid sequence of SEQ ID NO: 8. In some embodiments, the heavy chain comprises the amino acid sequence of SEQ ID NO: 9. In some embodiments, the light chain comprises the amino acid sequence of SEQ ID NO: 10.

Further provided herein is a polynucleotide comprising a nucleotide sequence encoding a heavy chain variable region comprising a CDRH1 domain comprising the amino acid sequence of SEQ ID NO: 11, a CDRH2 domain comprising the amino acid sequence of SEQ ID NO: 12, and a CDRH3 domain comprising the amino acid sequence of SEQ ID NO: 13; and a light chain variable region comprising a CDRL1 domain comprising the amino acid sequence of SEQ ID NO: 14, a CDRL2 domain comprising the amino acid sequence of SEQ ID NO: 15, and a CDRL3 domain comprising the amino acid sequence of SEQ ID NO: 16. In some embodiments, the heavy chain variable region comprises the amino acid sequence of SEQ ID NO: 17. In some embodiments, the light chain variable region comprises the amino acid sequence of SEQ ID NO: 18. In some embodiments, the heavy chain comprises the amino acid sequence of SEQ ID NO: 19. In some embodiments, the light chain comprises the amino acid sequence of SEQ ID NO: 20.

In a further aspect, provided herein is a polynucleotide comprising a nucleotide sequence encoding a heavy chain variable region comprising a CDRH1 domain comprising the amino acid sequence of SEQ ID NO: 21, a CDRH2 domain comprising the amino acid sequence of SEQ ID NO: 22, and a CDRH3 domain comprising the amino acid sequence of SEQ ID NO: 23; and a light chain variable region comprising a CDRL1 domain comprising the amino acid sequence of SEQ ID NO: 24, a CDRL2 domain comprising the amino acid sequence of SEQ ID NO: 25, and a CDRL3 domain comprising the amino acid sequence of SEQ ID NO: 26. In some embodiments, the heavy chain variable region comprises the amino acid sequence of SEQ ID NO: 27. In some embodiments, the light chain variable region comprises the amino acid sequence of SEQ ID NO: 28. In some embodiments, the heavy chain comprises the amino acid sequence of SEQ ID NO: 29. In some embodiments, the light chain comprises the amino acid sequence of SEQ ID NO: 30.

An anti-SARS-COV-2 spike antibody of the disclosure is preferably prepared by recombinant expression of immunoglobulin light and heavy chain genes in a host cell. To express an antibody recombinantly, a host cell is transfected with one or more recombinant expression vectors carrying DNA fragments encoding the immunoglobulin light and heavy chains of the antibody such that the light and heavy chains are expressed in the host cell and, optionally, secreted into the medium in which the host cells are cultured, from which medium the antibodies can be recovered.

To generate polynucleotides encoding such anti-anti-SARS-COV-2 spike antibodies, DNA fragments encoding the light and heavy chain variable regions are first obtained. These DNAs can be obtained by amplification and modification of germline DNA or cDNA encoding light and heavy chain variable sequences, for example using the polymerase chain reaction (PCR).

Once DNA fragments encoding anti-SARS-COV-2 spike antibody-related VH and VL segments are obtained, these DNA fragments can be further manipulated by standard recombinant DNA techniques, for example to convert the variable region genes to full-length antibody chain genes, to Fab fragment genes or to a scFv gene. In these manipulations, a VL-or VH-encoding DNA fragment is operatively linked to another DNA fragment encoding another protein, such as an antibody constant region or a flexible linker. The term “operatively linked,” as used in this context, is intended to mean that the two DNA fragments are joined such that the amino acid sequences encoded by the two DNA fragments remain in-frame.

The isolated DNA encoding the VH region can be converted to a full-length heavy chain gene by operatively linking the VH-encoding DNA to another DNA molecule encoding heavy chain constant regions (CH1, CH2, CH3 and, optionally, CH4). The sequences of human heavy chain constant region genes are known in the art (See, e.g., Kabat, E. A., et al., 1991, Sequences of Proteins of Immunological Interest, Fifth Edition, U.S. Department of Health and Human Services, NIH Publication No. 91-3242) and DNA fragments encompassing these regions can be obtained by standard PCR amplification. The heavy chain constant region can be an IgG1, IgG2, IgG3, IgG4, IgA, IgE, IgM or IgD constant region, but in certain embodiments is an IgG1 or IgG4. For a Fab fragment heavy chain gene, the VH-encoding DNA can be operatively linked to another DNA molecule encoding only the heavy chain CH1 constant region.

The isolated DNA encoding the VL region can be converted to a full-length light chain gene (as well as a Fab light chain gene) by operatively linking the VL-encoding DNA to another DNA molecule encoding the light chain constant region, CL. The sequences of human light chain constant region genes are known in the art (See, e.g., Kabat, et al., 1991, Sequences of Proteins of Immunological Interest, Fifth Edition, U.S. Department of Health and Human Services, NIH Publication No. 91-3242) and DNA fragments encompassing these regions can be obtained by standard PCR amplification. The light chain constant region can be a kappa or lambda constant region, but in certain embodiments is a kappa constant region.

To express the anti-SARS-COV-2 spike antibodies of the disclosure, DNAs encoding partial or full-length light and heavy chains, obtained as described above, are inserted into expression vectors such that the genes are operatively linked to transcriptional and translational control sequences. In this context, the term “operatively linked” is intended to mean that an antibody gene is ligated into a vector such that transcriptional and translational control sequences within the vector serve their intended function of regulating the transcription and translation of the antibody gene. The expression vector and expression control sequences are chosen to be compatible with the expression host cell used. The antibody light chain gene and the antibody heavy chain gene can be inserted into separate vectors or, more typically, both genes are inserted into the same expression vector.

The antibody genes are inserted into the expression vector by standard methods (e.g., ligation of complementary restriction sites on the antibody gene fragment and vector, or blunt end ligation if no restriction sites are present). Prior to insertion of the anti-SARS-COV-2 spike antibody-related light or heavy chain sequences, the expression vector can already carry antibody constant region sequences. For example, one approach to converting the anti-SARS-COV-2 spike monoclonal antibody-related VH and VL sequences to full-length antibody genes is to insert them into expression vectors already encoding heavy chain constant and light chain constant regions, respectively, such that the VH segment is operatively linked to the CH segment(s) within the vector and the VL segment is operatively linked to the CL segment within the vector. Additionally or alternatively, the recombinant expression vector can encode a signal peptide that facilitates secretion of the antibody chain from a host cell. The antibody chain gene can be cloned into the vector such that the signal peptide is linked in-frame to the amino terminus of the antibody chain gene. The signal peptide can be an immunoglobulin signal peptide or a heterologous signal peptide (i.e., a signal peptide from a non-immunoglobulin protein).

In addition to the antibody chain genes, the recombinant expression vectors of the disclosure carry regulatory sequences that control the expression of the antibody chain genes in a host cell. The term “regulatory sequence” is intended to include promoters, enhancers, and other expression control elements (e.g., polyadenylation signals) that control the transcription or translation of the antibody chain genes.

In addition to the antibody chain genes and regulatory sequences, the recombinant expression vectors of the disclosure can carry additional sequences, such as sequences that regulate replication of the vector in host cells (e.g., origins of replication) and selectable marker genes. The selectable marker gene facilitates selection of host cells into which the vector has been introduced. For expression of the light and heavy chains, the expression vector(s) encoding the heavy and light chains is transfected into a host cell by standard techniques. The various forms of the term “transfection” are intended to encompass a wide variety of techniques commonly used for the introduction of exogenous DNA into a prokaryotic or eukaryotic host cell, e.g., electroporation, lipofection, calcium-phosphate precipitation, DEAE-dextran transfection and the like.

It is possible to express the antibodies of the disclosure in either prokaryotic or eukaryotic host cells. In certain embodiments, expression of antibodies is performed in eukaryotic cells, e.g., mammalian host cells, of optimal secretion of a properly folded and immunologically active antibody. Exemplary mammalian host cells for expressing the recombinant antibodies of the disclosure include Chinese Hamster Ovary (CHO cells) (including DHFR-CHO cells, described in Urlaub and Chasin, 1980, Proc. Natl. Acad. Sci. USA 77:4216-4220, used with a DHFR selectable marker, e.g., as described in Kaufman and Sharp, 1982, Mol. Biol. 159:601-621), NSO myeloma cells, COS cells and SP2 cells. When recombinant expression vectors encoding antibody genes are introduced into mammalian host cells, the antibodies are produced by culturing the host cells for a period of time sufficient to allow for expression of the antibody in the host cells or secretion of the antibody into the culture medium in which the host cells are grown. Antibodies can be recovered from the culture medium using standard protein purification methods. Host cells can also be used to produce portions of intact antibodies, such as Fab fragments or scFv molecules. It is understood that variations on the above procedure are within the scope of the present disclosure. For example, it can be desirable to transfect a host cell with DNA encoding either the light chain or the heavy chain (but not both) of an anti-SARS-COV-2 spike antibody of this disclosure.

Recombinant DNA technology can also be used to remove some or all of the DNA encoding either or both of the light and heavy chains that is not necessary for binding to a SARS-COV-2 spike protein. The molecules expressed from such truncated DNA molecules are also encompassed by the antibodies of the disclosure.

For recombinant expression of an anti-SARS-COV-2 spike antibody of the disclosure, the host cell can be co-transfected with two expression vectors of the disclosure, the first vector encoding a heavy chain derived polypeptide and the second vector encoding a light chain derived polypeptide. The two vectors can contain identical selectable markers, or they can each contain a separate selectable marker. Alternatively, a single vector can be used which encodes both heavy and light chain polypeptides.

Once a polynucleotide encoding one or more portions of an anti-SARS-COV-2 spike antibody has been obtained, further alterations or mutations can be introduced into the coding sequence, for example to generate polynucleotides encoding antibodies with different CDR sequences, antibodies with reduced affinity to the Fc receptor, or antibodies of different subclasses.

The anti-SARS-COV-2 spike antibodies of the disclosure can also be produced by chemical synthesis or by using a cell-free platform.

Once a polypeptide of the disclosure has been produced by recombinant expression, it can be isolated or purified by any method known in the art for purification of a protein.

The anti-SARS-COV-2 spike antibodies of this disclosure may be provided as a composition suitable for administration to a subject. In some embodiments, the composition comprises two anti-SARS-COV-2 spike antibodies (e.g., two antibodies having different CDRs and/or epitopes). In one embodiment, the composition comprises a non-ACE-2 competitive antibody and an ACE-2 competitive antibody.

In some embodiments, the non-ACE-2 competitive antibody of the composition binds to an epitope comprising three peptide regions of the spike protein: ¹²⁴DSKVGGNYNYL¹³⁴ (SEQ ID NO: 32), ¹⁵⁰ISTE¹⁵³ (SEQ ID NO: 33), and ¹⁷⁷YGF⁷⁹ (SEQ ID NO: 34). In some embodiments, the non-ACE-2 competitive antibody further binds to an epitope comprising the following peptide region of the spike protein: ⁹¹QIAPGQTGNIADYN¹⁰⁴ (SEQ ID NO: 35). See, for example, the epitope of TPP-14584 described in Example 3.

In some embodiments, the antibody is an ACE-2 competitive antibody. In one embodiment, the ACE-2 competitive antibody binds to an epitope comprising three peptide regions of the spike protein: 91QIAPGQTGNIADYN104 (SEQ ID NO: 40), ¹³⁶RLF¹³⁸ (SEQ ID NO: 41), and ¹⁵⁴IYQAGNKPCNGVAGENCYFP¹⁷³ (SEQ ID NO: 42).

In another embodiment, the ACE-2 competitive antibody binds to an epitope comprising three peptide regions of the spike protein: ³¹SVYAWNRKRISNCVAD⁴⁶ (SEQ ID NO: 36), ⁷⁵TNVYADSF⁸² (SEQ ID NO: 37), ¹³²NYL¹³⁴ (SEQ ID NO: 38), and ¹⁵⁰ISTE¹⁵³ (SEQ ID NO: 39).

In some embodiments, the composition comprises two anti-SARS-COV-2 spike antibodies selected from:
(i) an antibody including a heavy chain variable region comprising a CDR1 domain comprising the amino acid sequence of SEQ ID NO: 1, a CDR2 domain comprising the amino acid sequence of SEQ ID NO: 2, and a CDR3 domain comprising the amino acid sequence of SEQ ID NO:3; and a light chain variable region comprising a CDR1 domain comprising the amino acid sequence of SEQ ID NO: 4, a CDR2 domain comprising the amino acid sequence of SEQ ID NO: 5, and a CDR3 domain comprising the amino acid sequence of SEQ ID NO: 6;
(ii) an antibody including a heavy chain variable region comprising a CDR1 domain comprising the amino acid sequence of SEQ ID NO: 11, a CDR2 domain comprising the amino acid sequence of SEQ ID NO: 12, and a CDR3 domain comprising the amino acid sequence of SEQ ID NO: 13; and a light chain variable region comprising a CDR1 domain comprising the amino acid sequence of SEQ ID NO: 14, a CDR2 domain comprising the amino acid sequence of SEQ ID NO: 15, and a CDR3 domain comprising the amino acid sequence of SEQ ID NO: 16; and
(iii) an antibody including a heavy chain variable region comprising a CDR1 domain comprising the amino acid sequence of SEQ ID NO: 21, a CDR2 domain comprising the amino acid sequence of SEQ ID NO: 22, and a CDR3 domain comprising the amino acid sequence of SEQ ID NO: 23; and a light chain variable region comprising a CDR1 domain comprising the amino acid sequence of SEQ ID NO: 24, a CDR2 domain comprising the amino acid sequence of SEQ ID NO: 25, and a CDR3 domain comprising the amino acid sequence of SEQ ID NO: 26.

In some embodiments, the composition comprises two anti-SARS-COV-2 spike antibodies selected from:
(i) an antibody including a heavy chain variable region comprising the amino acid sequence of SEQ ID NO: 7 and a light chain variable region comprises the amino acid sequence of SEQ ID NO: 8;
(ii) an antibody including a heavy chain variable region comprising the amino acid sequence of SEQ ID NO: 17 and a light chain variable region comprising the amino acid sequence of SEQ ID NO: 18; and
(iii) an antibody including a heavy chain variable region comprising the amino acid sequence of SEQ ID NO: 27 and a light chain variable region comprises the amino acid sequence of SEQ ID NO: 28.

In some embodiments, the composition comprises two anti-SARS-COV-2 spike antibodies selected from:
(i) an antibody including a heavy chain comprising the amino acid sequence of SEQ ID NO: 9 and a light chain comprising the amino acid sequence of SEQ ID NO: 10;
(ii) an antibody including a heavy chain comprising the amino acid sequence of SEQ ID NO: 19 and a light chain comprising the amino acid sequence of SEQ ID NO: 20; and
(iii) an antibody including a heavy chain comprising the amino acid sequence of SEQ ID NO: 29 and a light chain comprising the amino acid sequence of SEQ ID NO: 30.

In one embodiment, provided herein is a composition comprising a first and a second antibody that specifically bind to an anti-SARS-COV-2 spike(S) protein, wherein (i) the first antibody comprises a heavy chain variable region comprising a CDR1 domain comprising the amino acid sequence of SEQ ID NO: 1, a CDR2 domain comprising the amino acid sequence of SEQ ID NO: 2, and a CDR3 domain comprising the amino acid sequence of SEQ ID NO:3; and a light chain variable region comprising a CDR1 domain comprising the amino acid sequence of SEQ ID NO: 4, a CDR2 domain comprising the amino acid sequence of SEQ ID NO: 5, and a CDR3 domain comprising the amino acid sequence of SEQ ID NO: 6; and (ii) the second antibody comprises a heavy chain variable region comprising a CDR1 domain comprising the amino acid sequence of SEQ ID NO: 21, a CDR2 domain comprising the amino acid sequence of SEQ ID NO: 22, and a CDR3 domain comprising the amino acid sequence of SEQ ID NO: 23; and a light chain variable region comprising a CDR1 domain comprising the amino acid sequence of SEQ ID NO: 24, a CDR2 domain comprising the amino acid sequence of SEQ ID NO: 25, and a CDR3 domain comprising the amino acid sequence of SEQ ID NO: 26.

In some embodiments, the first antibody comprises a heavy chain variable region comprising the amino acid sequence of SEQ ID NO: 7 and a light chain variable region comprises the amino acid sequence of SEQ ID NO: 8; and the second antibody comprises a heavy chain variable region comprising the amino acid sequence of SEQ ID NO: 27 and a light chain variable region comprises the amino acid sequence of SEQ ID NO: 28.

In some embodiments, the first antibody comprises a heavy chain comprising the amino acid sequence of SEQ ID NO: 9 and a light chain comprising the amino acid sequence of SEQ ID NO: 10; and the second antibody comprises a heavy chain comprising the amino acid sequence of SEQ ID NO: 29 and a light chain comprising the amino acid sequence of SEQ ID NO: 30. For example, in one embodiment, the composition includes TPP-16613 (ACE-2 Competitive) and TPP-14584 (non-ACE-2 competitive).

In another embodiment, provided herein is a composition comprising a first and a second antibody that specifically bind to an anti-SARS-COV-2 spike(S) protein, wherein (i) the first antibody comprises a heavy chain variable region comprising a CDR1 domain comprising the amino acid sequence of SEQ ID NO: 1, a CDR2 domain comprising the amino acid sequence of SEQ ID NO: 2, and a CDR3 domain comprising the amino acid sequence of SEQ ID NO: 3; and a light chain variable region comprising a CDR1 domain comprising the amino acid sequence of SEQ ID NO: 4, a CDR2 domain comprising the amino acid sequence of SEQ ID NO: 5, and a CDR3 domain comprising the amino acid sequence of SEQ ID NO: 6; and (ii) the second antibody comprises a heavy chain variable region comprising a CDR1 domain comprising the amino acid sequence of SEQ ID NO: 11, a CDR2 domain comprising the amino acid sequence of SEQ ID NO: 12, and a CDR3 domain comprising the amino acid sequence of SEQ ID NO: 13; and a light chain variable region comprising a CDR1 domain comprising the amino acid sequence of SEQ ID NO: 14, a CDR2 domain comprising the amino acid sequence of SEQ ID NO: 15, and a CDR3 domain comprising the amino acid sequence of SEQ ID NO: 16.

In some embodiments, the first antibody comprises a heavy chain variable region comprising the amino acid sequence of SEQ ID NO: 7 and a light chain variable region comprising the amino acid sequence of SEQ ID NO: 8; and the second antibody comprises a heavy chain variable region comprising the amino acid sequence of SEQ ID NO: 17 and a light chain variable region comprising the amino acid sequence of SEQ ID NO: 18.

In some embodiments, the first antibody comprises a heavy chain comprising the amino acid sequence of SEQ ID NO: 9 and a light chain comprising the amino acid sequence of SEQ ID NO: 10; and the second antibody comprises a heavy chain comprising the amino acid sequence of SEQ ID NO: 19 and a light chain comprising the amino acid sequence of SEQ ID NO: 20. In one embodiment, the composition includes TPP-16613 (ACE-2 Competitive) and TPP-16124 (non-ACE-2 competitive).

In some embodiments, the composition comprises at least two anti-SARS-COV-2 spike antibodies (e.g., two, three, four, or more antibodies having different CDRs and/or epitopes).

### Therapeutic Uses

Additionally provided are methods of treating or preventing an infection by a SARS-COV-2 virus in a subject, preferably a human subject, using an anti-SARS-COV-2 spike(S) antibody, or antigen-binding fragment thereof, of the present disclosure. The antibodies can inhibit or neutralize SARS-COV-2 activity, and thus can be used for treating or preventing diseases associated with a SARS-COV-2 infection (e.g., COVID-19) in humans. The method can either be a prophylactic treatment or a therapeutic treatment measure.

In some embodiments, the method is a prophylactic treatment to control, maintain, or reduce levels of a SARS-COV-2 virus or at least one symptom associated with such an infection by a SARS-COV-2 virus. In some embodiments, the method involves prophylactically treating or preventing an infection by a SARS-COV-2 virus by administering an effective amount of an anti-SARS-COV-2 spike(S) antibody disclosed herein to a subject in need thereof. Subjects in need of prophylactic treatment may include, for example, those vulnerable to or at high risk of SARS-COV-2 viral infections (e.g., immunocompromised subjects or subjects at high risk of exposure) and/or subjects at risk of symptoms (e.g., moderate to severe symptoms) associated with SARS-COV-2 viral infection. Preventative or prophylactic measures include measures taken before a known exposure to a SARS-COV-2 virus (i.e., pre-exposure prophylactic) or measures taken after a known exposure to a SARS-COV-2 virus (i.e., post-exposure prophylactic).

In one embodiment, the method is a therapeutic treatment to control, maintain, or reduce levels of a SARS-COV-2 virus or at least one symptom associated with such an infection by a SARS-COV-2 virus (e.g., to treat, lessen one or more symptoms of, and/or slow or halt progression of a diagnosed SARS-COV-2 infection). In some embodiments, the methods involve therapeutically treating an infection by a SARS-COV-2 virus by administering an effective amount of an anti-SARS-COV-2 spike(S) antibody of the present disclosure to a subject in need thereof.

In accordance with the present disclosure, compositions herein may be administered to a mammalian subject, preferably a human. Administration may be in a therapeutically effective amount, which is an amount sufficient to show benefit to the subject (e.g., an immunocompromised subject or an individual at risk of infection). Therapeutic efficacy by an anti-SARS-COV-2 spike protein antibody can be measured, for example, based on a viral titer in a biological sample from the subject (e.g., a sample from the nasal cavity, lung, or throat of the subject) and/or one or alleviation or prevention of one or more symptoms associated with the SARS-COV-2 infection. Methods of measuring viral titer are known in the art (e.g., coronavirus propagation in embryonated chicken eggs or coronavirus spike protein assay).

In certain embodiments, the subject is an immunocompromised subject. Immunocompromised subjects have a reduced or weakened immune system due to one or more defects or dysfunctions of the immune system and/or due to other factors that heighten susceptibility of the subject to an infection, such as treatment with an immunosuppressive agent. The subject may be immunocompromised due to a primary and secondary immunodeficiency. Examples of primary immunodeficiencies include, but are not limited to, common variable immunodeficiency (CVID), chronic granulomatous disease (CGD), severe combined immunodeficiency (SCID), X-linked agammaglobulinemia, Wiskott-Aldrich syndrome, DiGeorge syndrome, or ataxia-telangiectasia. Examples of secondary immunodeficiencies include, but are not limited to, acquired immunodeficiency syndrome (AIDS) or a hematological malignancy, such as leukemia, lymphoma, or multiple myeloma. In some embodiments, the subject is immunocompromised due to treatment with an immunosuppressive agent, such as a chemotherapeutic agent, a biologic agent (e.g., anti-tumor necrosis factor), a steroid (e.g., a corticosteroid), an antimetabolite (e.g., mycophenolate), or a lymphodepleting agent (e.g., anti-CD20 antibodies). In some embodiments, the subject has undergone a solid organ transplant or a stem cell transplant. In some embodiments, the subject has an age-associated immune deficiency (i.e., immunosenescence).

The antibodies described herein can also be used to treat or prevent coronavirus disease 2019 (COVID-19) caused by severe acute respiratory syndrome coronavirus 2 (SARS-COV-2) in individuals 12 years of age and older.

Further, the disclosed anti-SARS-COV-2 spike antibodies, can be administered individually or in combination with an additional anti-SARS-COV-2 spike antibody or fragments thereof, as further described in the present disclosure.

Further aspects and embodiments of the disclosure will be apparent to those skilled in the art given the present disclosure including the following experimental exemplification.

### EXAMPLES

The following Examples may be used for illustrative purposes and should not be deemed to narrow the scope of the invention.

### Example 1. Generation of Anti-SARS-COV-2 Antibodies

The following Example describes the generation of recombinant anti-SARS-CoV-2 antibodies that bind to conserved regions of the SARS-COV-2 spike protein. First, a library of antibodies that target different epitopes on the SARS-COV-2 virus was created by screening B cell repertoires of patients who have had a SARS-COV-2 infection, patients who were previously administered a SARS-COV-2 vaccine, or patients who had a breakthrough infection. Variable regions of SARS-COV-2 antibodies identified in the screen were then selected for modification and incorporation into recombinant antibodies for further characterization.

Methods-Generation of anti SARS COV 2 antibodies

Anti SARS COV2 monoclonal antibodies were isolated from human donors who were actively infected, recovered and/or vaccinated for SARS COV2. Plasma cells were isolated from PBMCs based on their selectivity to SARS COV2 recombinant antigen. Single B cells were screened for binding to SARS COV2 ECD and RBS antigen as described in Winters et al, Crowe et al. More than a 100,000 B cells were screened from each human donor with multiple donors screened. B cells secreting antibodies specific for antigen were isolated and sequenced.

Recover immunoglobulin gene sequences from SARS-COV-2-specific single B cell by NGS and variable region sequence analysis

To generate recombinant antibody from the immunoglobin gene of the SARS-CoV-2-specific single B cell for downstream characterization, the heavy and light chain variable regions are captured by RT-PCR and next generation sequencing (NGS). After a serial of bioinformatics processing of the NGS data, the identified full-length heavy and light chain paired sequences are cloned into the expression vectors containing the constant region of immunoglobulin gene for transfection of mammalian cells to produce recombinant antibodies. After the full-length variable region sequences are identified from the NGS data, the germline names were assigned for each sequence by the germline library.

### Example 2: Characterization of Anti-SARS-COV-2 Antibodies

The following Example describes the in vitro characterization of recombinant anti-SARS-COV-2 antibodies identified in Example 1. The neutralization activity of each antibody was assessed individually and in combination (see Example 3). A pseudovirus library containing all SARS-COV-2 variants of concern was generated to evaluate the neutralization potential of the three mAbs of interest (TPP-16613, TPP-16124, and TPP-14584).

In vitro PsV neutralization data against SARS-COV-2 variants and SARS-COV-1

The pseudovirus is frequently used for measuring the effectiveness of antibodies. In this study, we used a replication-incompetent lentivirus coated with spike proteins from SARS-COV-2 Wuhan-Hu-1 strain, various SARS-COV-2 variants of concerns/emerging variants under monitoring, or SARS-COV-1 to evaluate a library of monoclonal antibodies targeting SARS-COV-2 spike protein.

### Methods

SARS-COV-2-S pseudovirus was generated by co-transfection of 293T cells with the spike expression construct and the pNL4-3. Luc. R-E-lentiviral backbone plasmid containing the firefly luciferase reporter gene. SARS-COV-2-S pseudoviruses were titered using VeroE6-TMPRSS2 cells, and the virus dilution corresponding to 25,000 RLUs was selected to evaluate activity of monoclonal antibody (mAb) in the neutralizing antibody assay. For the neutralizing antibody assay, mAb was evaluated at a starting concentration of 30.0 ug/mL using a 10-point dilution series with 4-fold serial dilutions, resulting in an assay range of 30.0-0.00011 ug/mL of mAb. Serial dilutions of mAb were incubated with SARS-COV-2-S pseudovirus for 1 hour at 37° C., followed by the addition of VeroE6-TMPRSS2 target cells. Assay plates were then incubated for 72 hours at 37° C. followed by luciferase measurements. Assay conditions were evaluated in triplicate. Neutralization activity was assessed by calculating percent inhibition of the luciferase signal in the presence of antibody relative to pseudovirus alone. The IC50, IC80, and IC90 values were calculated using the nonlinear regression curve fitting to the 4-parameter logistic equation in GraphPad Prism 8/9 software.

SARS-COV-2-S pseudovirus was purified by ultracentrifugation and spike was detected in the purified virions. The neutralization profile of SOC mAb such as Sotrovimab and Bebtelovimab determined using our pseudoviruses matched that using authentic SARS-COV-2 variants. These suggest that SARS-COV-2-S pseudovirus is qualified for evaluating the effectiveness of mAbs.

Results for Individual mAbs

The neutralization potential of the three mAbs of interest against SARS-COV-2 pseudovirus generated from various recent variants was determined. The neutralization of each mAb was assessed via inhibition of SARS-COV-2 pseudovirus infection (measured by luciferase activity). The IC50 values generated from each data set are shown in Table 1 (all are reported as ng/ml). TPP-14584 and TPP-16613 exhibited potent neutralization of each of the tested SARS-COV-2 pseudoviruses with high potency (IC50 values ranging in the single to triple digit ng/ml range). Variants to which one of the mAbs had EC50 greater than 30,000 ng/ml include SARS1, BF.7, BA4.6, BQ.1.1, BA.2.86, HV.1, JN.1 XBB1.5.70, GK.2, JD.1.1 and JF.1. Of note, TPP-16124, showed potent neutralization against SARS-COV-1 (IC50 value is 28 ng/ml). Between the three antibodies, they could neutralize all SARS COV2 variants of concern.

TABLE 1
mAb neutralization IC50 for SARS-CoV-2-S pseudovirus

| | TPP-14584 | | TPP-16613 | | | | Omi42 | | S309 | |
|---|---|---|---|---|---|---|---|---|---|---|
| | (ABBV-2412) | | (ABBV-1403) | | TPP-16124 | | (AZD3152) | | (Sotrovimab) | |
| | IC50 | IC80 | IC50 | IC80 | IC50 | IC80 | IC50 | IC80 | IC50 | IC80 |
| | (ng/ml) | (ng/ml) | (ng/ml) | (ng/ml) | (ng/ml) | (ng/ml) | (ng/ml) | (ng/ml) | (ng/ml) | (ng/ml) |
| SARS1 | >30,000 | >30,000 | >30,000 | >30,000 | 28 | 562 | >30,000 | >30,000 | 2 | 12 |
| Urbani | | | | | | | | | | |
| Wuhan | 1 | 5 | 20 | 36 | 52 | 220 | 37 | 62 | 5 | 25 |
| D614G | | | | | | | | | | |
| Alpha | 1 | 7 | 2 | 31 | 33 | 120 | 6 | 41 | 13 | 158 |
| Beta | 3 | 18 | 2 | 28 | 140 | 1,161 | 5 | 46 | 10 | 57 |
| Delta | 5 | 13 | 20 | 38 | 92 | 183 | 48 | 109 | 10 | 25 |
| Gamma | 6 | 18 | 2 | 8 | 46 | 79 | 2 | 21 | 12 | 51 |
| Omicron | 5 | 21 | 4 | 11 | 275 | 1,062 | 12 | 39 | 59 | 663 |
| BA.1 | | | | | | | | | | |
| BA.2 | 12 | 22 | 7 | 62 | 183 | 208 | 20 | 240 | 357 | 2,575 |
| BA.4/5 | 3 | 15 | 7 | 21 | 395 | 2,396 | 20 | 43 | 1,200 | 8,537 |
| BA.2.75 | 10 | 27 | 9 | 41 | 97 | 510 | 22 | 88 | 332 | 19,500 |
| BF.7 | >30,000 | >30,000 | 7 | 23 | 237 | 529 | 20 | 49 | 2,012 | >30,000 |
| BA.4.6 | >30,000 | >30,000 | 8 | 27 | 173 | 542 | 23 | 53 | 2,148 | >30,000 |
| BJ.1 | 30 | 82 | 8 | 32 | 621 | 1,796 | 21 | 67 | 8,139 | >30,000 |
| XBB | 19 | 118 | 22 | 58 | 739 | 2,252 | 16 | 43 | 53 | 384 |
| XBB.1 | 38 | 174 | 2 | 12 | 375 | 931 | 2 | 15 | 632 | 14,718 |
| BQ.1 | 21 | 142 | 34 | 82 | 359 | 1,204 | 60 | 125 | 419 | 3,312 |
| BQ.1.1 | >30,000 | >30,000 | 27 | 76 | 813 | 3,349 | 39 | 100 | 2,385 | >30,000 |
| XBB.1.5 | 108 | 369 | 18 | 47 | 1,282 | 2,811 | 13 | 38 | 357 | 4,396 |
| XBB.1.16 | 52 | 140 | 4 | 19 | 935 | 2,449 | 18 | 36 | 264 | 1,515 |
| XBB.1.16.1 | 52 | 262 | 4 | 20 | 726 | 3,485 | 13 | 65 | 385 | 2,333 |
| XBB.1.16.6 | 65 | 238 | 854 | 2,466 | 488 | 1,931 | 6,396 | 21,923 | 244 | 982 |
| XBB.2.3 | 61 | 253 | 4 | 17 | 518 | 1,436 | 9 | 31 | 433 | 3,915 |
| EG.5.1 | 62 | 256 | 796 | 2,022 | 472 | 1,428 | 6,225 | 16,740 | 370 | 2,605 |
| FL.1.5.1 | 49 | 347 | 917 | 3,413 | 337 | 3,751 | 7,892 | 27,820 | 477 | 5,815 |
| BA.2.86 | >30,000 | >30,000 | 2 | 16 | >30,000 | >30,000 | 8 | 50 | 6,423 | >30,000 |
| HV.1 | >30,000 | >30,000 | 752 | 2,301 | 253 | 738 | 3,763 | >30,000 | 238 | 1,178 |
| XBB.1.5.70 | 98 | 369 | >30,000 | >30,000 | 1,167 | 3,902 | 16,056 | >30,000 | 266 | 2,754 |
| GK.2 | 132 | 420 | >30,000 | >30,000 | 931 | 2,440 | 8,212 | >30,000 | 203 | 696 |
| HK.3 | 188 | 407 | 13,920 | >30,000 | 1,299 | 2,561 | NA | NA | NA | NA |
| JD.1.1 | 189 | 601 | >30,000 | >30,000 | 1,073 | 2,208 | >30,000 | >30,000 | 541 | 2,965 |
| JF.1 | 95 | 275 | >30,000 | >30,000 | 1,193 | 2,870 | 3,643 | 13,420 | 425 | 3,102 |
| JN.1 | >30,000 | >30,000 | 5 | 34 | >30,000 | >30,000 | 64 | 310 | 6,623 | >30,000 |

### Example 3: Characterization of Combinations of Anti-SARS-COV-2 Antibodies

In this Example, the neutralization activity against SARS-COV-2 variants of each antibody in Example 2 was assessed in combination.

### Results for mAb Combination

The neutralization potential of two mAbs cocktail was further tested against SARS-COV-2 BQ. 1, BQ. 1.1, XBB, and XBB.1.5 pseudovirus. The neutralization was assessed via inhibition of SARS-COV-2 pseudovirus infection (measured by luciferase activity). The IC50 values generated from each data set are shown in Table 2 (all are reported as ng/ml). As shown above in Table 1, Antibody-3 (TPP-14584) alone failed to neutralize BQ.1.1 pseudovirus while Antibody-2 (TPP-16613) neutralized BQ.1.1 pseudovirus (IC50 value is 3 ng/ml). However, supplementing Antibody-3 (TPP-14584) with Antibody-2 (TPP-16613) rescued its failure in neutralizing BQ. 1.1 pseudovirus. Both mAb combinations, Antibody-2 (TPP-16613)+Antibody-3 (TPP-14584) and Antibody-2 (TPP-16613)+Antibody-4 (TPP-16124), neutralized all four variant pseudoviruses with IC50 values ranging from 76-526 ng/ml. These results suggest mAb combinations allow for a panel approach to tackling the diverse variants of concern (VOC) that may arise from SARS-COV-2.

TABLE 2
mAb cocktail neutralization IC50 for SARS-CoV-2-S pseudovirus

| SARS- | | | | | |
|---|---|---|---|---|---|
| CoV-2 | | | | | |
| Variants | Single mAb | | | Combination mAbs | |
| IC50 | Antibody-2 | Antibody-3 | Antibody-4 | Antibody-2 + | Antibody-2 + |
| (ng/mL) | (TPP-16613) | (TPP-14584) | (TPP-16124) | Antibody 3 | Antibody-4 |
| BQ.1 | 4 | 11 | 290 | 76 | 141 |
| BQ.1.1 | 3 | >30,000 | 760 | 50 | 152 |
| XBB | 1 | 23 | 784 | 17 | 86 |
| XBB.1.5 | 18 | 144 | 973 | 158 | 526 |

### Example 4: Epitope Mapping of Anti-SARS-COV-2 Antibodies

The following Example describes the epitope mapping of the three lead recombinant anti-SARS-COV-2 antibodies identified in Examples 1 and 2: TPP-14584, TPP-16124, and TPP-16124.

### Material and Methods

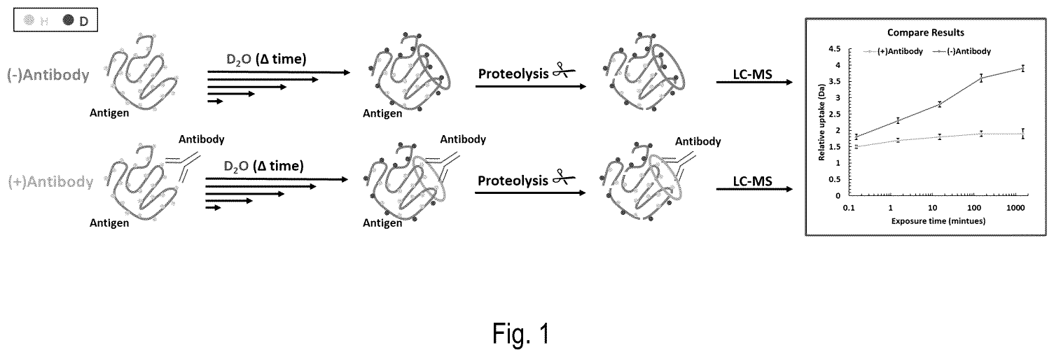

Hydrogen-Deuterium Exchange Mass Spectrometry (HDX-MS) is an MS based technique that can be used to characterize interactions between an antigen-antibody complex and identify the epitope interface in solution. This approach has been widely used in epitope mapping of protein therapeutics due to its unique features, including universal labeling properties, high sensitivity and robustness. HDX-MS relies on the exchange of protein backbone amide hydrogen atoms with deuterium atoms in solution. To identify binding sites, HDX-MS experiments are generally performed on both free antibody and antigen-antibody complex. The data collected from the two experiments is compared, and regions within the protein that possess different deuterium incorporation are considered to define the epitope of the antibody. A typical experimental design of HDX-MS epitope mapping is shown in FIG. 1.

In this experiment, the HDX-MS epitope mapping technique was used to identify the epitopes of three antibodies. SARS-COV-2 receptor binding domain (RBD) was chosen as the target protein.

### HDX-MS Experiment

All HDX-MS experiments were performed using a nanoACQUITY UPLC system with HDX technology (Waters Corporation, Milford, MA). For free-antigen HDX experiments, the concentration of SARS-COV-2 BA2 RBD protein was adjusted to 17.5 μM using 1X PBS (pH 7.4). For the antigen-antibody complex experiments, SARS-COV-2 BA2 RBD protein (17.5 μM) was mixed with TPP-14584, TPP-16613, or TPP-16124 using a molar ratio of 1 to 1.1 in 1×PBS (pH 7.4). The mixtures were incubated at 25° C. for 30 min to ensure complete complex formation. In the non-deuterated experiment, aliquots of 6 μL of the samples were diluted with 54 μL aqueous equilibration buffer (10 mM sodium phosphate buffer, pH 7.2) at 20° C. in duplicates. For HDX experiments, aliquots of 6 μL of the samples were diluted with 54 μL labeling buffer (10 mM sodium phosphate buffer, D₂O, pD 7.2) in triplicates at 20° C. for different periods of time: 15 s, 3 min, and 30 min. At the end of each labeling reaction, 50 μL of the sample was quenched in 50 μL of quench buffer (100 mM sodium phosphate, 4 M guanidine hydrochloride and 1 M TCEP, pH 2.4) and incubated for 2 min at 1° C. A 100 UL aliquot of each quenched sample was injected into a nanoACQUITY UPLC system equipped with an Enzymate™ BEH pepsin column (2.1 mm×30 mm, Waters Corporation, Milford, MA) for online digestion at 15° C. The desalting and separation were carried out at 0.2° C. using a BEH C18 VanGaurd™ pre-column (2.1 mm×5 mm, 1.7 μm, Waters Corporation, Milford, MA) and an ACQUITY BEH C18 analytical column (1.0 mm×100 mm, 1.7 μm, Waters Corporation, Milford, MA). Peptides were separated using a 12 min linear acetonitrile gradient (5-40%) containing 0.1% formic acid at 40 μL/min. The eluent was directly sprayed into a Synapt G2 mass spectrometer (Waters Corporation, Milford, MA) with electrospray ionization and lock-mass correction using Leu-Enkephalin solution. Mass spectra of non-deuterated free SARS-COV-2 BA2 RBD protein were acquired in MSE mode with ion mobility over an m/z range of 275 to 2000 for peptide identification. Mass spectra of all other samples were acquired in MS mode with ion mobility over an m/z range of 275 to 2000 for improved spectra quality. The sample cone voltage was set to 30 V and the transfer collision energy was set to ramp from 15-35 V. Blanks of 0.1% formic acid in water were injected in between each LC-MS run using the same analytical gradient to minimize the carryovers.

### HDX-MS Data Analysis

All peptides were identified from non-deuteration controls using ProteinLynx Global Server 3.0.2 software (Waters Corporation, Milford, MA). The level of deuterium uptake for each peptide was first using DynamX 3.0 software (Waters Corporation, Milford, MA). After the automated analysis, the HDX assignment of each peptide was evaluated and confirmed. A single charge state that contained high quality spectra for all replicates across all HDX labeling time points in SARS-COV-2 BA2 RBD protein and all complex samples was selected to represent HDX for each peptide. The level of deuterium uptake for each peptide was determined based on the difference between the un-deuterated and deuterated samples using DynamX 3.0 software (Waters Corporation). The deuterium uptake difference was determined by comparing the level of deuterium uptake of free antigen and antigen-Fab/antibody complex samples. Potential epitopes were determined for each antibody using a deuterium uptake difference cut-off of 0.5 Da for a single time point. All spectra and data processing were manually confirmed and validated for epitope identification.

### Results

The HDX-MS experiment was used to identify the epitope of the three test antibodies. Significant deuterium uptake differences were determined by comparing the HDX uptake plots of antigen and antigen-antibody complex samples. The results suggest each of the tested antibodies targets a unique epitope on the receptor binding domain (RBD) of the SARS-COV-2 spike protein. First, the epitope of TPP-14584 includes three peptide regions: ¹²⁴DSKVGGNYNYL¹³⁴ (SEQ ID NO: 32), ¹⁵⁰ISTE¹⁵³ (SEQ ID NO: 33), and ¹⁷⁷YGF⁷⁹ (SEQ ID NO: 34). In addition, minor HDX reduction was observed in the peptide region of ⁹¹QIAPGQTGNIADYN¹⁰⁴ (SEQ ID NO: 35). This indicates that local solvent protection occurs in this region upon the binding of TPP-14584. Second, the epitope of TPP-16124 includes four peptide regions: ³¹SVYAWNRKRISNCVAD⁴⁶ (SEQ ID NO: 36), ⁷⁵TNVYADSF⁸² (SEQ ID NO: 37), ¹³²NYL¹³⁴ (SEQ ID NO: 38), and ¹⁵⁰ISTE¹⁵³ (SEQ ID NO: 39). Finally, the epitope of TPP-16613 includes three peptide regions: ⁹¹QIAPGQTGNIADYN¹⁰⁴ (SEQ ID NO: 40), ¹³⁶RLF¹³⁸ (SEQ ID NO: 41), and ¹⁵⁴IYQAGNKPCNGVAGENCYFP¹⁷³ (SEQ ID NO: 42).

### Example 5: Surface Plasmon Resonance (SPR) Biacore Binding Studies for Lead Anti-SARS-COV-2 Antibodies

The following Example describes an in vitro antigen binding study for the following three lead recombinant anti-SARS-COV-2 antibodies identified in Examples 1 and 2: TPP-14584, TPP-16124, and TPP-16124.

Material and Methods for SPR Biacore Binding Studies for Lead Anti SARS-COV-2 mAbs

A Biacore T200 instrument, with Biacore T200 control software Version 4.0.1, from Cytiva Inc. (formerly GE Healthcare), Marlborough MA, was used to run kinetic experiments with SARS-COV-2 RBD soluble protein variants. Experimental data from the Biacore T200 instrument was analyzed using Biacore evaluation software Version 4.1, GE Healthcare Life Sciences.

Antibody binding was assessed against the following SARS-COV-2 variant RBDs:
Recombinant SARS-COV2 (319-540) S protein RBD WT, 0.79 mg/ml, 29617 Da
Recombinant SARS-COV-2 (319-540) RBD, Omicron BA.1.1.529, 0.6 mg/ml, 26800 Da
Recombinant SARS-COV-2 (319-540) RBD, Omicron BA.2, 1.01 mg/ml, 27245 Da
Recombinant SARS-COV-2 (319-540) RBD, Omicron BA.2.75, 1.75 mg/ml, 27283 Da
Recombinant SARS-COV-2 (319-540) RBD, Omicron BA.3, 0.2 mg/ml, 26800 Da
Recombinant SARS-COV-2 (319-540) RBD, Omicron BA.4/5, 0.4 mg/ml, 26700 Da
Recombinant SARS-COV-2 (319-540) RBD, Omicron BA.4.6, 1.19 mg/ml, 27000 Da.
Recombinant SARS-COV-2 (319-540) RBD, Omicron BA.4.6 V445A, 1.16 mg/ml, 27000 Da
Recombinant SARS-COV-2 (319-540) RBD, Omicron XBB, 0.9 mg/ml, 27106 Da
Recombinant SARS-COV-2 (319-540) RBD, Omicron BQ1.1, 0.81 mg/ml, 27144 Da
Recombinant SARS-COV-1 RBD, 2.08 mg/ml, 26000 Da
Recombinant Human ACE2 receptor, 2.6 mg/ml, 176524 Da

### Preparation of Biosensor Surfaces

Goat antibodies specific to the Fc region of human IgG were used as capture for Anti SARS-COV-2 mAbs. They were covalently linked to the carboxy methyl dextran matrix on the CM5 biosensor chip (Cytiva, catalog no. BR-1006-68) via free amine groups using an amine coupling kit from Cytiva (Catalog no. BR-1000-50). Carboxyl groups of the dextran matrix on the chip were activated with 100 mM NHS and 400 mM EDC. Goat anti-human IgG Fc (25 ug/mL; Invitrogen, catalog no. 31125), diluted in 10 mM sodium acetate, pH 4.5 (Cytiva, catalog no. BR-1003-50), was injected across all four of the activated flow cell surfaces. Once the level of binding response reached the desired value, unreacted groups were deactivated by injection of 1 M ethanolamine. Approximately 9000-10000 RU of goat anti-human IgG Fc was immobilized on the each of the four active flow cell surfaces.

Binding of Anti SARS-COV-2 Lead mAbs to Recombinant SARS-COV-2 RBD Variants

Anti SARS-COV-2 lead mAbs, diluted to 1 μg/mL in HBS-EP 1x (10 mM HEPES buffer pH 7.4 containing 150 mM NaCl, 3 mM EDTA, 0.005% P20), were injected over the goat anti-human IgG Fc surfaces, at a flow rate of 10 μL/min for different contact times (30s-180s) to achieve capture level of 100-150 RU. The net difference in the baseline signal and the signal after the completion of the antibody injections were taken to represent the amount of captured mAbs. For the antigen (recombinant SARS-COV-2 RBD variants) binding experiments it consisted of antigen association and antigen dissociation phases, all experiments were run at 25° C. temperature. Aliquots of recombinant SARS-COV-2 RBD variants were injected at different concentrations at a flow rate of 50 μL/min for 5 min over captured Anti SARS-COV-2 mAbs to ascertain association rates. Recombinant SARS-COV-2 RBD variants were tested at the following concentrations: 0, 0.48, 2.4, 12, 60, and 300 nM. Dissociation phase consisted of continued flow of buffer containing, at 50 μL/min for 10 min. The instrument responses were measured in RU and are proportional to the mass of bound recombinant SARS-CoV-2 RBD variants. Reaction surfaces were regenerated with two consecutive pulses of 25 μL injections of 10 mM glycine (pH 1.5; Cytiva, catalog no. BR-1003-54) at a flow rate of 50 μL/min, before the injection of the next sample. The reference surface responses, with no captured antibodies, were subtracted from the reaction surface data, to eliminate changes in the refractive index and injection noise.

Results for Anti SARS-COV-2 Lead mAbs Binding to Recombinant SARS-COV-2 RBD Variants

Kinetic rate parameters, such as on-rate and off-rate and affinity for Anti SARS-CoV-2 Mabs binding to recombinant SARS COV-2 RBD variants, are summarized in Table 3. Data was fit by a 1:1 binding model.

As shown in Table 3, overall, affinity of lead Anti SARS-COV-2 mAbs, in nanomolar, was superior compared to control competitor mAbs.

TABLE 3
Lead and Competitor Anti SARS-CoV-2 mAbs

| | Anti SARS-CoV-2 Lead/ | | | | TPP-16613 LS, AC-376859 | | | TPP-14584 LS, AC-355148 | | |
|---|---|---|---|---|---|---|---|---|---|---|
| | Competitor mAbs | | | | PR-2206316, 3045366 | | | PR-2151197, 3053673 | | |
| | Biologic Naming | | PR- # | Lot # | kₐ (1/Ms) | k_{d} (1/s) | K_{D} (M) | kₐ (1/Ms) | k_{d} (1/s) | K_{D} (M) |
| 1 | SARS-CoV-2 | Wuha- | PR- | 2673618 | 7.3E+05 | 2.3E−05 | 3.1E−11 | 1.4E+06 | 1.5E−05 | 1.1E−11 |
| | RBD WT | Wu-1 | 1969017 | | | | | | | |
| 2 | SARS-CoV-2 | Omicron | PR- | 2861563 | 1.5E+06 | 1.7E−04 | 1.2E−10 | 1.5E+06 | 1.4E−06 | 8.8E−13 |
| | RBD | | 2113299 | | | | | | | |
| | BA.1.1.529 | | | | | | | | | |
| 3 | SARS-CoV-2 | Omicron | PR- | 2887466 | 1.3E+06 | 1.8E−04 | 1.4E−10 | 3.0E+06 | 6.6E−06 | 2.2E−12 |
| | RBD BA.2 | | 2129120 | | | | | | | |
| 4 | SARS-CoV-2 | Omicron | PR- | 2917486 | 1.5E+06 | 4.2E−04 | 2.8E−10 | 1.4E+06 | 1.2E−05 | 8.4E−12 |
| | RBD BA.2.75 | | 2156729 | | | | | | | |
| 5 | SARS-CoV-2 | Omicron | PR- | 3056558 | 1.8E+06 | 3.1E−04 | 1.7E−10 | | | No Binding |
| | RBD BA2.86 | | 2252818 | | | | | | | |
| 6 | SARS-CoV-2 | Omicron | PR- | 2900364 | 2.4E+06 | 7.0E−05 | 2.9E−11 | 2.9E+06 | 1.5E−05 | 4.9E−12 |
| | RBD BA.3 | | 2142064 | | | | | | | |
| 7 | SARS-CoV-2 | Omicron | PR- | 2903754 | 1.9E+06 | 7.4E−05 | 4.0E−11 | 2.0E+06 | 1.5E−04 | 7.8E−11 |
| | RBD BA.4/5 | | 2145435 | | | | | | | |
| 8 | SARS-CoV-2 | Omicron | PR- | 2971247 | 1.3E+06 | 7.6E−05 | 6.0E−11 | 5.1E+05 | 4.8E−04 | 9.4E−10 |
| | RBD BA4.6 | | 2183962 | | | | | | | |
| 9 | SARS-CoV-2 | Omicron | PR- | 2971248 | 1.5E+06 | 7.5E−05 | 5.2E−11 | 5.3E+05 | 4.8E−04 | 9.1E−10 |
| | RBD BA4.6 | | 2183964 | | | | | | | |
| | V445A | | | | | | | | | |
| 10 | SARS-CoV-2 | Omicron | PR- | 2973966 | 1.2E+06 | 2.4E−04 | 2.1E−10 | | | No Binding |
| | RBD BQ1.1 | | 2187578 | | | | | | | |
| 11 | SARS-CoV-2 | Omicron | PR- | 2973965 | 2.2E+06 | 3.2E−04 | 1.5E−10 | 4.4E+05 | 1.1E−03 | 2.4E−09 |
| | RBD XBB | | 2187577 | | | | | | | |
| 12 | SARS-CoV-2 | Omicron | PR- | 3025918 | 2.0E+06 | 1.8E−04 | 8.9E−11 | 4.5E+05 | 1.1E−03 | 2.5E−09 |
| | RBD XBB | | 2226073 | | | | | | | |
| | 1.1.6 | | | | | | | | | |
| 13 | SARS-CoV-2 | Omicron | PR- | 3051080 | 1.9E+05 | 5.5E−04 | 2.9E−09 | 6.1E+05 | 1.3E−03 | 2.1E−09 |
| | RBD XBB 1.16 | | 2248037 | | | | | | | |
| | L455W | | | | | | | | | |
| 14 | SARS-CoV-2 | Omicron | PR- | 3042712 | 2.3E+06 | 1.9E−04 | 8.1E−11 | 4.8E+05 | 1.2E−03 | 2.4E−09 |
| | RBD XBB 2.3 | | 2242432 | | | | | | | |
| 15 | SARS-CoV-2 | Omicron | PR- | 3051551 | 1.5E+05 | 5.6E−04 | 3.7E−09 | 5.8E+05 | 1.3E−03 | 2.2E−09 |
| | RBD XBB 2.3 | | 2248038 | | | | | | | |
| | L455W | | | | | | | | | |
| 16 | SARS-CoV-2 | Omicron | PR- | 3044798 | 4.3E+04 | 8.7E−04 | 2.0E−08 | 4.5E+05 | 1.4E−03 | 3.1E−09 |
| | RBD EG.5.1 | | 2244001 | | | | | | | |
| 17 | SARS-CoV-2 | Omicron | PR- | 3051081 | | | No Binding | 4.7E+05 | 1.4E−03 | 3.0E−09 |
| | RBD EG.5.1 | | 2248033 | | | | | | | |
| | L455W | | | | | | | | | |
| 18 | SARS-CoV-2 | Omicron | PR- | 3053097 | 7.0E+04 | 1.1E−03 | 1.5E−08 | 4.1E+05 | 1.4E−03 | 3.3E−09 |
| | RBD FL.1.5.1 | | 2249520 | | | | | | | |
| 19 | SARS-CoV-2 | Omicron | PR- | 3053098 | 2.6E+06 | 2.8E−04 | 1.1E−10 | 5.1E+05 | 1.2E−03 | 2.3E−09 |
| | RBD EU.1.1 | | 2249519 | | | | | | | |
| 20 | SARS-CoV-2 | Omicron | PR- | 3061007 | 5.8E+04 | 1.1E−03 | 1.9E−08 | 3.6E+04 | 6.3E−04 | 1.8E−08 |
| | RBD HV.1 | | 2256698 | | | | | | | |
| 21 | SARS-CoV-2 | Omicron | PR- | 3067337 | | | No Binding | 2.9E+05 | 1.4E−03 | 4.8E−09 |
| | RBD HK.3 | | 2262133 | | | | | | | |
| 22 | SARS-CoV-2 | Omicron | PR- | 3078550 | 9.3E+05 | 6.1E−04 | 6.5E−10 | | | No Binding |
| | RBD JD.1.1 | | 2271451 | | | | | | | |
| 23 | SARS-CoV-2 | Omicron | PR- | 3078551 | 1.6E+06 | 6.3E−04 | 3.8E−10 | | | No Binding |
| | RBD JN.1 | | 2271455 | | | | | | | |
| 24 | SARS-CoV-1 | Urbani | PR- | 2672745 | | | No Binding | | | No Binding |
| | RBD CoV-1 | | 1982709 | | | | | | | |
| | WT | | | | | | | | | |
| | ACE2 | | PR- | 2651578 | Blocking | | | Non-Blocking | | |
| | Competition | | 1966966 | | | | | | | |
| | profile | | | | | | | | | |

| | Anti SARS-CoV-2 Lead/ | | | | TPP-16124 LS, AC-365971 | | | AZD-3152, AstraZeneca | | |
|---|---|---|---|---|---|---|---|---|---|---|
| | Competitor mAbs | | | | PR-2208877, 3014689 | | | PR-2246604, 3046613 | | |
| | Biologic Naming | | PR- # | Lot # | kₐ (1/Ms) | k_{d} (1/s) | K_{D} (M) | kₐ (1/Ms) | k_{d} (1/s) | K_{D} (M) |
| 1 | SARS-CoV-2 | Wuha- | PR- | 2673618 | 8.4E+05 | 1.2E−05 | 1.4E−11 | 2.3E+05 | 9.9E−06 | 4.2E−11 |
| | RBD WT | Wu-1 | 1969017 | | | | | | | |
| 2 | SARS-CoV-2 | Omicron | PR- | 2861563 | 2.2E+06 | 4.0E−06 | 1.9E−12 | 6.7E+05 | 5.4E−05 | 8.1E−11 |
| | RBD | | 2113299 | | | | | | | |
| | BA.1.1.529 | | | | | | | | | |
| 3 | SARS-CoV-2 | Omicron | PR- | 2887466 | 1.9E+06 | 6.0E−06 | 3.2E−12 | 4.7E+05 | 6.4E−05 | 1.4E−10 |
| | RBD BA.2 | | 2129120 | | | | | | | |
| 4 | SARS-CoV-2 | Omicron | PR- | 2917486 | 1.7E+06 | 1.0E−06 | 5.8E−13 | 4.0E+05 | 1.1E−03 | 2.7E−09 |
| | RBD BA.2.75 | | 2156729 | | | | | | | |
| 5 | SARS-CoV-2 | Omicron | PR- | 3056558 | 1.8E+05 | 8.1E−04 | 4.6E−09 | 5.0E+05 | 2.4E−03 | 4.8E−09 |
| | RBD BA2.86 | | 2252818 | | | | | | | |
| 6 | SARS-CoV-2 | Omicron | PR- | 2900364 | 3.5E+06 | 1.0E−06 | 2.8E−13 | 9.0E+05 | 4.1E−05 | 4.6E−11 |
| | RBD BA.3 | | 2142064 | | | | | | | |
| 7 | SARS-CoV-2 | Omicron | PR- | 2903754 | 2.7E+06 | 2.8E−06 | 1.0E−12 | 1.0E+06 | 3.3E−05 | 3.2E−11 |
| | RBD BA.4/5 | | 2145435 | | | | | | | |
| 8 | SARS-CoV-2 | Omicron | PR- | 2971247 | 1.2E+06 | 1.6E−06 | 1.3E−12 | 5.9E+05 | 3.9E−05 | 6.6E−11 |
| | RBD BA4.6 | | 2183962 | | | | | | | |
| 9 | SARS-CoV-2 | Omicron | PR- | 2971248 | 1.3E+06 | 1.0E−06 | 7.7E−13 | 5.4E+05 | 3.7E−05 | 6.8E−11 |
| | RBD BA4.6 | | 2183964 | | | | | | | |
| | V445A | | | | | | | | | |
| 10 | SARS-CoV-2 | Omicron | PR- | 2973966 | 8.3E+05 | 1.2E−06 | 1.4E−12 | 4.9E+05 | 1.6E−03 | 3.3E−09 |
| | RBD BQ1.1 | | 2187578 | | | | | | | |
| 11 | SARS-CoV-2 | Omicron | PR- | 2973965 | 7.2E+05 | 1.0E−06 | 1.4E−12 | 5.9E+05 | 1.2E−03 | 2.1E−09 |
| | RBD XBB | | 2187577 | | | | | | | |
| 12 | SARS-CoV-2 | Omicron | PR- | 3025918 | 8.1E+05 | 1.7E−06 | 2.1E−12 | 7.7E+05 | 9.4E−04 | 1.2E−09 |
| | RBD XBB | | 2226073 | | | | | | | |
| | 1.1.6 | | | | | | | | | |
| 13 | SARS-CoV-2 | Omicron | PR- | 3051080 | 1.4E+06 | 1.0E−06 | 7.1E−13 | 1.7E+05 | 6.2E−04 | 3.6E−09 |
| | RBD XBB 1.16 | | 2248037 | | | | | | | |
| | L455W | | | | | | | | | |
| 14 | SARS-CoV-2 | Omicron | PR- | 3042712 | 9.2E+05 | 3.8E−06 | 4.1E−12 | 9.6E+05 | 1.1E−03 | 1.1E−09 |
| | RBD XBB 2.3 | | 2242432 | | | | | | | |
| 15 | SARS-CoV-2 | Omicron | PR- | 3051551 | 1.3E+06 | 1.1E−06 | 8.5E−13 | 1.7E+05 | 9.0E−04 | 5.3E−09 |
| | RBD XBB 2.3 | | 2248038 | | | | | | | |
| | L455W | | | | | | | | | |
| 16 | SARS-CoV-2 | Omicron | PR- | 3044798 | 8.6E+05 | 3.0E−06 | 3.5E−12 | | | No Binding |
| | RBD EG.5.1 | | 2244001 | | | | | | | |
| 17 | SARS-CoV-2 | Omicron | PR- | 3051081 | 9.6E+05 | 1.0E−06 | 1.0E−12 | | | No Binding |
| | RBD EG.5.1 | | 2248033 | | | | | | | |
| | L455W | | | | | | | | | |
| 18 | SARS-CoV-2 | Omicron | PR- | 3053097 | 8.4E+05 | 4.8E−06 | 5.7E−12 | | | No Binding |
| | RBD FL.1.5.1 | | 2249520 | | | | | | | |
| 19 | SARS-CoV-2 | Omicron | PR- | 3053098 | 1.1E+06 | 3.4E−06 | 3.3E−12 | 1.1E+06 | 1.8E−03 | 1.6E−09 |
| | RBD EU.1.1 | | 2249519 | | | | | | | |
| 20 | SARS-CoV-2 | Omicron | PR- | 3061007 | 1.4E+06 | 6.1E−06 | 4.5E−12 | | | No Binding |
| | RBD HV.1 | | 2256698 | | | | | | | |
| 21 | SARS-CoV-2 | Omicron | PR- | 3067337 | 6.8E+05 | 1.5E−05 | 2.2E−11 | | | No Binding |
| | RBD HK.3 | | 2262133 | | | | | | | |
| 22 | SARS-CoV-2 | Omicron | PR- | 3078550 | 1.2E+05 | 5.1E−04 | 4.4E−09 | 2.5E+05 | 1.2E−02 | 4.8E−08 |
| | RBD JD.1.1 | | 2271451 | | | | | | | |
| 23 | SARS-CoV-2 RBD | Omicron | PR- | 3078551 | 1.9E+05 | 4.9E−04 | 2.5E−09 | 3.5E+05 | 9.7E−03 | 2.8E−08 |
| | JN.1 | | 2271455 | | | | | | | |
| 24 | SARS-CoV-1 RBD | Urbani | PR- | 2672745 | 4.7E+05 | 4.0E−03 | 8.5E−09 | | | No Binding |
| | CoV-1 WT | | 1982709 | | | | | | | |
| | ACE2 Competition | | PR- | 2651578 | Non-Blocking | | | Blocking | | |
| | profile | | 1966966 | | | | | | | |

| | Anti SARS-CoV-2 Lead/ | | | | Bebtelovimab, Lilly/AbCellera | | | S309, GSK/VIR | | |
|---|---|---|---|---|---|---|---|---|---|---|
| | Competitor mAbs | | | | PR-2134258, 2892056 | | | PR-1970409, 2650549 | | |
| | Biologic Naming | | PR- # | Lot # | kₐ (1/Ms) | k_{d} (1/s) | K_{D} (M) | kₐ (1/Ms) | k_{d} (1/s) | K_{D} (M) |
| 1 | SARS-CoV-2 RBD | Wuha- | PR- | 2673618 | 2.8E+05 | 3.6E−04 | 1.3E−09 | 4.5E+04 | 3.3E−05 | 7.3E−10 |
| | WT | Wu-1 | 1969017 | | | | | | | |
| 2 | SARS-CoV-2 RBD | Omicron | PR- | 2861563 | 4.7E+05 | 3.7E−03 | 7.8E−09 | 5.4E+04 | 1.9E−04 | 3.4E−09 |
| | BA.1.1.529 | | 2113299 | | | | | | | |
| 3 | SARS-CoV-2 RBD | Omicron | PR- | 2887466 | 1.1E+06 | 2.1E−04 | 1.9E−10 | | | NT |
| | BA.2 | | 2129120 | | | | | | | |
| 4 | SARS-CoV-2 RBD | Omicron | PR- | 2917486 | 5.1E+05 | 3.1E−03 | 6.1E−09 | 6.2E+04 | 1.6E−05 | 2.5E−10 |
| | BA.2.75 | | 2156729 | | | | | | | |
| 5 | SARS-CoV-2 RBD | Omicron | PR- | 3056556 | | | No Binding | | | Weak |
| | BA2.86 | | 2252818 | | | | | | | Binding |
| 6 | SARS-CoV-2 RBD | Omicron | PR- | 2900364 | 7.5E+05 | 2.9E−03 | 3.8E−09 | 1.2E+05 | 4.0E−04 | 3.4E−09 |
| | BA.3 | | 2142064 | | | | | | | |
| 7 | SARS-CoV-2 RBD | Omicron | PR- | 2903754 | 1.1E+06 | 2.9E−04 | 2.8E−10 | 5.0E+04 | 4.8E−04 | 9.6E−09 |
| | BA.4/5 | | 2145435 | | | | | | | |
| 8 | SARS-CoV-2 RBD | Omicron | PR- | 2971247 | 3.7E+05 | 1.6E−04 | 4.2E−10 | | | NT |
| | BA4.6 | | 2183962 | | | | | | | |
| 9 | SARS-CoV-2 RBD | Omicron | PR- | 2971248 | 5.6E+05 | 1.8E−02 | 3.3E−08 | | | NT |
| | BA4.6 V445A | | 2183964 | | | | | | | |
| 10 | SARS-CoV-2 RBD | Omicron | PR- | 2973966 | | | No Binding | 2.5E+04 | 5.9E−04 | 2.4E−08 |
| | BQ1.1 | | 2187578 | | | | | | | |
| 11 | SARS-CoV-2 RBD | Omicron | PR- | 2973965 | | | No Binding | 2.9E+04 | 6.3E−05 | 2.2E−09 |
| | XBB | | 2187577 | | | | | | | |
| 12 | SARS-CoV-2 RBD | Omicron | PR- | 3025918 | | | No Binding | 3.4E+04 | 6.2E−05 | 1.8E−09 |
| | XBB 1.1.6 | | 2226073 | | | | | | | |
| 13 | SARS-CoV-2 RBD | Omicron | PR- | 3051080 | | | No Binding | 5.7E+04 | 1.1E−04 | 1.9E−09 |
| | XBB 1.16 L455W | | 2248037 | | | | | | | |
| 14 | SARS-CoV-2 RBD | Omicron | PR- | 3042712 | | | No Binding | 4.2E+04 | 8.1E−05 | 1.9E−09 |
| | XBB 2.3 | | 2242432 | | | | | | | |
| 15 | SARS-CoV-2 RBD | Omicron | PR- | 3051551 | | | No Binding | 5.5E+04 | 1.3E−04 | 2.3E−09 |
| | XBB 2.3 L455W | | 2248038 | | | | | | | |
| 16 | SARS-CoV-2 RBD | Omicron | PR- | 3044798 | | | No Binding | 3.3E+04 | 6.0E−05 | 1.8E−09 |
| | EG.5.1 | | 2244001 | | | | | | | |
| 17 | SARS-CoV-2 RBD | Omicron | PR- | 3051081 | | | No Binding | 4.7E+04 | 1.3E−04 | 2.8E−09 |
| | EG.5.1 L455W | | 2248033 | | | | | | | |
| 18 | SARS-CoV-2 RBD | Omicron | PR- | 3053097 | | | No Binding | 4.2E+04 | 1.4E−04 | 3.3E−09 |
| | FL.1.5.1 | | 2249520 | | | | | | | |
| 19 | SARS-CoV-2 RBD | Omicron | PR- | 3053098 | | | No Binding | 5.1E+04 | 1.3E−04 | 2.6E−09 |
| | EU.1.1 | | 2249519 | | | | | | | |
| 20 | SARS-CoV-2 RBD | Omicron | PR- | 3061007 | | | No Binding | 2.6E+04 | 2.9E−05 | 1.1E−09 |
| | HV.1 | | 2256698 | | | | | | | |
| 21 | SARS-CoV-2 RBD | Omicron | PR- | 3067337 | | | No Binding | 2.3E+04 | 1.1E−04 | 4.7E−09 |
| | HK.3 | | 2262133 | | | | | | | |
| 22 | SARS-CoV-2 RBD | Omicron | PR- | 3078550 | | | No Binding | | | Weak |
| | JD.1.1 | | 2271451 | | | | | | | Binding |
| 23 | SARS-CoV-2 RBD | Omicron | PR- | 3078551 | | | No Binding | | | Weak |
| | JN.1 | | 2271455 | | | | | | | Binding |
| 24 | SARS-CoV-1 RBD | Urbani | PR- | 2672745 | | | No Binding | 8.6E+04 | 2.6E−05 | 3.0E−10 |
| | CoV-1 WT | | 1982709 | | | | | | | |
| | ACE2 Competition | | PR- | 2651578 | Blocking | | | Non-Blocking | | |
| | profile | | 1966966 | | | | | | | |

SEQUENCE SUMMARY TABLE

| SEQ ID | | |
|---|---|---|
| NO | DESCRIPTION | AMINO ACID SEQUENCE |
| 1 | TPP-16613-COV Antibody | GFTLSSFGIH |
| | CDR-H1 | |
| 2 | TPP-16613-COV Antibody | VIWYDGSDQHYADSVKG |
| | CDR-H2 | |
| 3 | TPP-16613-CoV Antibody | DLWPLLAVYYGMDV |
| | CDR-H3 | |
| 4 | TPP-16613-COV Antibody | RASQSVSSYLA |
| | CDR-L1 | |
| 5 | TPP-16613-COV Antibody | EASNRAT |
| | CDR-L2 | |
| 6 | TPP-16613-CoV Antibody | QQRSNWPYT |
| | CDR-L3 | |
| 7 | TPP-16613-CoV Antibody | EMQLVESGGGVVQPGASLRLSCTASGF |
| | Heavy Chain Variable | TLSSFGIHWVRQAPGKGLEWVAVIWYD |
| | Region (VH) | GSDQHYADSVKGRFTVSRDNSKNIVYL |
| | | QMDSLRAEDTALYYCAKDLWPLLAVYY |
| | | GMDVWGQGTTVTVSS |
| 8 | TPP-16613-CoV Antibody | EIVLTQSPATLSLSPGKRATLSCRASQ |
| | Light Chain Variable | SVSSYLAWYQQKPGQAPRLLIYEASNR |
| | Region (VL) | ATGIPDRFSGSGSETDFTLTISSLEPE |
| | | DFAVYYCQQRSNWPYTFGQGTKLEIE |
| 9 | TPP-16613-CoV Antibody | EMQLVESGGGVVQPGASLRLSCTASGE |
| | Heavy Chain - Full | TLSSFGIHWVRQAPGKGLEWVAVIWYD |
| | Length (Variable | GSDQHYADSVKGRFTVSRDNSKNIVYL |
| | region underlined) | QMDSLRAEDTALYYCAKDLWPLLAVYY |
| | | GMDVWGQGTTVTVSSASTKGPSVFPLA |
| | | PSSKSTSGGTAALGCLVKDYFPEPVTV |
| | | SWNSGALTSGVHTFPAVLQSSGLYSLS |
| | | SVVTVPSSSLGTQTYICNVNHKPSNTK |
| | | VDKKVEPKSCDKTHTCPPCPAPELLGG |
| | | PSVFLFPPKPKDTLMISRTPEVTCVVV |
| | | DVSHEDPEVKFNWYVDGVEVHNAKTKP |
| | | REEQYNSTYRVVSVLTVLHQDWLNGKE |
| | | YKCKVSNKALPAPIEKTISKAKGQPRE |
| | | PQVYTLPPSREEMTKNQVSLTCLVKGF |
| | | YPSDIAVEWESNGQPENNYKTTPPVLD |
| | | SDGSFFLYSKLTVDKSRWQQGNVFSCS |
| | | VLHEALHSHYTQKSLSLSPGK |
| 10 | TPP-16613-COV Antibody | EIVLTQSPATLSLSPGKRATLSCRASQ |
| | Light Chain - Full | SVSSYLAWYQQKPGQAPRLLIYEASNR |
| | Length (Variable | ATGIPDRFSGSGSETDFTLTISSLEPE |
| | region underlined) | DFAVYYCQQRSNWPYTFGQGTKLEIER |
| | | TVAAPSVFIFPPSDEQLKSGTASVVCL |
| | | LNNFYPREAKVQWKVDNALQSGNSQES |
| | | VTEQDSKDSTYSLSSTLTLSKADYEKH |
| | | KVYACEVTHQGLSSPVTKSFNRGEC |
| 11 | TPP-16124-COV Antibody | GFMFSDSGLH |
| | CDR-H1 | |
| 12 | TPP-16124-COV Antibody | HIRSAPNNYATAYGESVRG |
| | CDR-H2 | |
| 13 | TPP-16124-COV Antibody | NDYLDYYQYGMDV |
| | CDR-H3 | |
| 14 | TPP-16124-CoV Antibody | RASQSINGWLA |
| | CDR-L1 | |
| 15 | TPP-16124-CoV Antibody | RASNLET |
| | CDR-L2 | |
| 16 | TPP-16124-CoV Antibody | QQYNSYSYT |
| | CDR-L3 | |
| 17 | TPP-16124-CoV Antibody | EVQLVESGGGLVQPGGSLKLSCAASGF |
| | Heavy Chain Variable | MFSDSGLHWVRQPSGKGLEWVGHIRSA |
| | Region (VH) (CDRs in | PNNYATAYGESVRGRFTIAIDESKNTA |
| | bold) | YLQMNSLKPEDTAVYFCTRNDYLDYYQ |
| | | YGMDVWGQGTTVTVSS |
| 18 | TPP-16124-CoV Antibody | DIQMTQSPSTLSASVGDRVIITCRASQ |
| | Light Chain Variable | SINGWLAWYQQKPGKAPNLLIYRASNL |
| | Region (VL) (CDRs in | ETGVPSRFSGSGSGTEFTLTISSLQPD |
| | bold) | DFATYYCQQYNSYSYTFGQGTKLESK |
| 19 | TPP-16124-COV Antibody | EVQLVESGGGLVQPGGSLKLSCAASGE |
| | Heavy Chain - Full | MFSDSGLHWVRQPSGKGLEWVGHIRSA |
| | Length (Variable | PNNYATAYGESVRGRFTIAIDESKNTA |
| | region underlined) | YLQMNSLKPEDTAVYFCTRNDYLDYYQ |
| | | YGMDVWGQGTTVTVSSASTKGPSVFPL |
| | | APSSKSTSGGTAALGCLVKDYFPEPVT |
| | | VSWNSGALTSGVHTFPAVLQSSGLYSL |
| | | SSVVTVPSSSLGTQTYICNVNHKPSNT |
| | | KVDKKVEPKSCDKTHTCPPCPAPELLG |
| | | GPSVFLFPPKPKDTLMISRTPEVTCVV |
| | | VDVSHEDPEVKFNWYVDGVEVHNAKTK |
| | | PREEQYNSTYRVVSVLTVLHQDWLNGK |
| | | EYKCKVSNKALPAPIEKTISKAKGQPR |
| | | EPQVYTLPPSREEMTKNQVSLTCLVKG |
| | | FYPSDIAVEWESNGQPENNYKTTPPVL |
| | | DSDGSFFLYSKLTVDKSRWQQGNVFSC |
| | | SVLHEALHSHYTQKSLSLSPGK |
| 20 | TPP-16124-CoV Antibody | DIQMTQSPSTLSASVGDRVIITCRASQ |
| | Light Chain - Full | SINGWLAWYQQKPGKAPNLLIYRASNL |
| | Length (Variable | ETGVPSRFSGSGSGTEFTLTISSLQPD |
| | region underlined) | DFATYYCQQYNSYSYTFGQGTKLESKR |
| | | TVAAPSVFIFPPSDEQLKSGTASVVCL |
| | | LNNFYPREAKVQWKVDNALQSGNSQES |
| | | VTEQDSKDSTYSLSSTLTLSKADYEKH |
| | | KVYACEVTHQGLSSPVTKSFNRGEC |
| 21 | TPP-14584-COV Antibody | GFTVRPNYMS |
| | CDR-H1 | |
| 22 | TPP-14584-COV Antibody | TLYRSGNTHYADSVKG |
| | CDR-H2 | |
| 23 | TPP-14584-CoV Antibody | ARVDYGDYFEDAFDV |
| | CDR-H3 | |
| 24 | TPP-14584-COV Antibody | RASQSISYFLN |
| | CDR-L1 | |
| 25 | TPP-14584-CoV Antibody | AASSLOS |
| | CDR-L2 | |
| 26 | TPP-14584-COV Antibody | QQSYNSLRIT |
| | CDR-L3 | |
| 27 | TPP-14584-COV Antibody | EVQLVESGGGLIQPGGSLRLSCAASGF |
| | Heavy Chain Variable | TVRPNYMSWVRQAPGKGLEWVSTLYRS |
| | Region (VH) (CDRs in | GNTHYADSVKGRFTISRDNSKNTLFLQ |
| | bold) | MNSLRAEDTAVYFCATARVDYGDYFED |
| | | AFDVWGQGTMVTVSS |
| 28 | TPP-14584-COV Antibody | DIQMTQSPSSLSASVGDRVTITCRASQ |
| | Light Chain Variable | SISYFLNWYQQKPGKAPKLLIFAASSL |
| | Region (VL) (CDRs in | QSGVPSRFSGSGSGTDFSLTIRGLQPE |
| | bold) | DFATYYCQQSYNSLRITFGQGTRLEIK |
| 29 | TPP-14584-COV Antibody | EVQLVESGGGLIQPGGSLRLSCAASGF |
| | Heavy Chain - Full | TVRPNYMSWVRQAPGKGLEWVSTLYRS |
| | Length (Variable | GNTHYADSVKGRFTISRDNSKNTLFLQ |
| | region underlined) | MNSLRAEDTAVYFCATARVDYGDYFED |
| | | AFDVWGQGTMVTVSSASTKGPSVFPLA |
| | | PSSKSTSGGTAALGCLVKDYFPEPVTV |
| | | SWNSGALTSGVHTFPAVLQSSGLYSLS |
| | | SVVTVPSSSLGTQTYICNVNHKPSNTK |
| | | VDKKVEPKSCDKTHTCPPCPAPELLGG |
| | | PSVFLFPPKPKDTLMISRTPEVTCVVV |
| | | DVSHEDPEVKFNWYVDGVEVHNAKTKP |
| | | REEQYNSTYRVVSVLTVLHQDWLNGKE |
| | | YKCKVSNKALPAPIEKTISKAKGQPRE |
| | | PQVYTLPPSREEMTKNQVSLTCLVKGF |
| | | YPSDIAVEWESNGQPENNYKTTPPVLD |
| | | SDGSFFLYSKLTVDKSRWQQGNVFSCS |
| | | VLHEALHSHYTQKSLSLSPGK |
| 30 | TPP-14584-COV Antibody | DIQMTQSPSSLSASVGDRVTITCRASQ |
| | Light Chain - Full | SISYFLNWYQQKPGKAPKLLIFAASSL |
| | Length (Variable | QSGVPSRFSGSGSGTDFSLTIRGLQPE |
| | region underlined) | DFATYYCQQSYNSLRITFGQGTRLEIK |
| | | RTVAAPSVFIFPPSDEQLKSGTASVVC |
| | | LLNNFYPREAKVQWKVDNALQSGNSQE |
| | | SVTEQDSKDSTYSLSSTLTLSKADYEK |
| | | HKVYACEVTHQGLSSPVTKSFNRGEC |
| 31 | SARS-COV-2 Spike | MFVFLVLLPLVSSQCVNLTTRTQLPPA |
| | Protein (RBD domain, | YTNSFTRGVYYPDKVFRSSVLHSTQDL |
| | corresponding to | FLPFFSNVTWFHAIHVSGTNGTKRFDN |
| | residues 319-541, is | PVLPFNDGVYFASTEKSNIIRGWIFGT |
| | underlined and bolded) | TLDSKTQSLLIVNNATNVVIKVCEFQF |
| | | CNDPFLGVYYHKNNKSWMESEFRVYSS |
| | | ANNCTFEYVSQPFLMDLEGKQGNFKNL |
| | | REFVFKNIDGYFKIYSKHTPINLVRDL |
| | | PQGFSALEPLVDLPIGINITRFQTLLA |
| | | LHRSYLTPGDSSSGWTAGAAAYYVGYL |
| | | QPRTFLLKYNENGTITDAVDCALDPLS |
| | | ETKCTLKSFTVEKGIYQTSNFRVQPTE |
| | | SIVRFPNITNLCPFGEVENATRFASVY |
| | | AWNRKRISNCVADYSVLYNSASFSTFK |
| | | CYGVSPTKLNDLCFTNVYADSFVIRGD |
| | | EVRQIAPGQTGKIADYNYKLPDDFTGC |
| | | VIAWNSNNLDSKVGGNYNYLYRLFRKS |
| | | NLKPFERDISTEIYQAGSTPCNGVEGF |
| | | NCYFPLQSYGFQPTNGVGYQPYRVVVL |
| | | SFELLHAPATVCGPKKSTNLVKNKCVN |
| | | FNFNGLTGTGVLTESNKKFLPFQQFGR |
| | | DIADTTDAVRDPQTLEILDITPCSFGG |
| | | VSVITPGTNTSNQVAVLYQDVNCTEVP |
| | | VAIHADQLTPTWRVYSTGSNVFQTRAG |
| | | CLIGAEHVNNSYECDIPIGAGICASYQ |
| | | TQTNSPRRARSVASQSIIAYTMSLGAE |
| | | NSVAYSNNSIAIPTNFTISVTTEILPV |
| | | SMTKTSVDCTMYICGDSTECSNLLLQY |
| | | GSFCTQLNRALTGIAVEQDKNTQEVFA |
| | | QVKQIYKTPPIKDFGGFNFSQILPDPS |
| | | KPSKRSFIEDLLFNKVTLADAGFIKQY |
| | | GDCLGDIAARDLICAQKFNGLTVLPPL |
| | | LTDEMIAQYTSALLAGTITSGWTFGAG |
| | | AALQIPFAMQMAYRENGIGVTQNVLYE |
| | | NQKLIANQFNSAIGKIQDSLSSTASAL |
| | | GKLQDVVNQNAQALNTLVKQLSSNFGA |
| | | ISSVLNDILSRLDKVEAEVQIDRLITG |
| | | RLQSLQTYVTQQLIRAAEIRASANLAA |
| | | TKMSECVLGQSKRVDFCGKGYHLMSFP |
| | | QSAPHGVVFLHVTYVPAQEKNFTTAPA |
| | | ICHDGKAHFPREGVFVSNGTHWFVTQR |
| | | NFYEPQIITTDNTFVSGNCDVVIGIVN |
| | | NTVYDPLQPELDSFKEELDKYFKNHTS |
| | | PDVDLGDISGINASVVNIQKEIDRLNE |
| | | VAKNLNESLIDLQELGKYEQYIKWPWY |
| | | IWLGFIAGLIAIVMVTIMLCCMTSCCS |
| | | CLKGCCSCGSCCKFDEDDSEPVLKGVK |
| | | LHYT |
| 32 | TPP-14584 Epitope - | DSKVGGNYNYL |
| | SARS-CoV-2 Spike | |
| | Protein - Region 1 | |
| 33 | TPP-14584 Epitope - | ISTE |
| | SARS- CoV-2 Spike | |
| | Protein - Region 2 | |
| 34 | TPP-14584 Epitope - | YGF |
| | SARS- CoV-2 Spike | |
| | Protein - Region 3 | |
| 35 | TPP-14584 Epitope - | QIAPGQTGNIADYN |
| | SARS-CoV-2 Spike | |
| | Protein - Region 4 | |
| 36 | TPP-16124 Epitope - | SVYAWNRKRISNCVAD |
| | SARS- CoV-2 Spike | |
| | Protein - Region 1 | |
| 37 | TPP-16124 Epitope - | TNVYADSF |
| | SARS- CoV-2 Spike | |
| | Protein - Region 2 | |
| 38 | TPP-16124 Epitope - | NYL |
| | SARS-CoV-2 Spike | |
| | Protein - Region 3 | |
| 39 | TPP-16124-Epitope - | ISTE |
| | SARS-CoV-2 Spike | |
| | Protein - Region 4 | |
| 40 | TPP-16613 Epitope - | QIAPGQTGNIADYN |
| | SARS- CoV-2 Spike | |
| | Protein - Region 1 | |
| 41 | TPP-16613 Epitope - | RLF |
| | SARS-CoV-2 Spike | |
| | Protein -Region 2 | |
| 42 | TPP-16613 Epitope - | IYQAGNKPCNGVAGENCYFP |
| | SARS-CoV-2 Spike | |
| | Protein - Region 3 | |

What is claimed is:

1. A monoclonal antibody that specifically binds to an anti-SARS-COV-2 spike(S) protein, wherein the antibody comprises
a heavy chain variable region comprising a CDRH1 domain comprising the amino acid sequence of SEQ ID NO: 1, a CDRH2 domain comprising the amino acid sequence of SEQ ID NO: 2, and a CDRH3 domain comprising the amino acid sequence of SEQ ID NO:3; and
a light chain variable region comprising a CDRL1 domain comprising the amino acid sequence of SEQ ID NO: 4, a CDRL2 domain comprising the amino acid sequence of SEQ ID NO: 5, and a CDRL3 domain comprising the amino acid sequence of SEQ ID NO: 6.

2. The monoclonal antibody of claim 1, wherein the heavy chain variable region comprises the amino acid sequence of SEQ ID NO: 7.

3. The monoclonal antibody of claim 1 wherein the light chain variable region comprises the amino acid sequence of SEQ ID NO: 8.

4. The monoclonal antibody of claim 1 wherein the heavy chain comprises the amino acid sequence of SEQ ID NO: 9.

5. The monoclonal antibody of claim 1, wherein the light chain comprises the amino acid sequence of SEQ ID NO: 10.

6. A monoclonal antibody that specifically binds to an anti-SARS-COV-2 spike(S) protein, wherein the antibody comprises
a heavy chain variable region comprising a CDRH1 domain comprising the amino acid sequence of SEQ ID NO: 11, a CDRH2 domain comprising the amino acid sequence of SEQ ID NO: 12, and a CDRH3 domain comprising the amino acid sequence of SEQ ID NO: 13; and
a light chain variable region comprising a CDRL1 domain comprising the amino acid sequence of SEQ ID NO: 14, a CDRL2 domain comprising the amino acid sequence of SEQ ID NO: 15, and a CDRL3 domain comprising the amino acid sequence of SEQ ID NO: 16.

7. The monoclonal antibody of claim 6, wherein the heavy chain variable region comprises the amino acid sequence of SEQ ID NO: 17.

8. The monoclonal antibody of claim 6, wherein the light chain variable region comprises the amino acid sequence of SEQ ID NO: 18.

9. The monoclonal antibody of claim 6 wherein the heavy chain comprises the amino acid sequence of SEQ ID NO: 19.

10. The monoclonal antibody of claim 6, wherein the light chain comprises the amino acid sequence of SEQ ID NO: 20.

11. A monoclonal antibody that specifically binds to an anti-SARS-COV-2 spike(S) protein, wherein the antibody comprises
a heavy chain variable region comprising a CDRH1 domain comprising the amino acid sequence of SEQ ID NO: 21, a CDRH2 domain comprising the amino acid sequence of SEQ ID NO: 22, and a CDRH3 domain comprising the amino acid sequence of SEQ ID NO: 23; and
a light chain variable region comprising a CDRL1 domain comprising the amino acid sequence of SEQ ID NO: 24, a CDRL2 domain comprising the amino acid sequence of SEQ ID NO: 25, and a CDRL3 domain comprising the amino acid sequence of SEQ ID NO: 26.

12. The monoclonal antibody of claim 11, wherein the heavy chain variable region comprises the amino acid sequence of SEQ ID NO: 27 and a light chain variable region comprises the amino acid sequence of SEQ ID NO: 28.

13. The monoclonal antibody of claim 11, comprising a heavy chain comprising the amino acid sequence of SEQ ID NO: 29 and a light chain comprising the amino acid sequence of SEQ ID NO: 30.

14. A pharmaceutical composition comprising the monoclonal antibody of claim 1.

15. A pharmaceutical composition comprising the monoclonal antibody of claim 11.

16. A pharmaceutical composition comprising the monoclonal antibody of claim 6.

17. The pharmaceutical composition of claim 14 for use in treating a SARS-COV-2 infection.

18. A method of controlling levels of a SARS-COV-2 virus in a subject comprising administering an effective amount of monoclonal antibody of claim 1 to a human subject in need thereof.

19. A method of controlling levels of a SARS-COV-2 virus in a human subject comprising administering an effective amount of the pharmaceutical composition of claim 14 to a human subject in need thereof.

20. A method of reducing and maintaining low levels of a SARS-COV-2 virus in a human subject comprising administering an effective amount of at least one monoclonal antibody of claim 1 to a human subject in need thereof.
